(19) United States
(12) Patent Application Publication
(10) Pub. No.: US 2026/0207894 A1
(43) Pub. Date: Jul. 23, 2026

(54) CATHETER INCLUDING NONINFLATABLE POSITIONING MEMBER

(71) Applicant: Medtronic Ireland Manufacturing Unlimited Company, Dublin (IE)

(72) Inventors: Brian J. Kelly, Oranmore (IE); Daniel M. King, Galway (IE); Tomas K. Kelly, Galway (IE)

(21) Appl. No.: 19/140,446

(22) PCT Filed: Dec. 12, 2023
(86) PCT No.: PCT/EP2023/085402

§ 371 Date: Jun. 18, 2025

Related U.S. Application Data
(60) Provisional application No. 63/476,974, filed on Dec. 23, 2022.

(51) Int. Cl. A61M 25/01; A61M 25/00

(52) U.S. Cl. CPC A61M 25/0158; A61M 25/007; A61M 25/0074; A61M 2210/12

(57) ABSTRACT

In some examples, a catheter (108, 300, 400, 500, 600, 700, 800, 900, 1000, 1100) comprises a catheter body (110), one or more noninflatable positioning members (116, 316, 416, 406, 516, 606, 716, 806, 816, 916, 1016, 1116) configured to deploy from a collapsed configuration to an expanded configuration, and one or more injection tubes (114, 314, 414, 514, 614, 714, 814, 914, 1014, 1114) connected to the one or more noninflatable positioning members. Each of the injection tubes defines an injection tube lumen and one or more injection ports (113, 313, 413, 513, 613, 713, 912 912B, 1013, 1113) in fluid communication with the respective injection tube lumen. The one or more noninflatable positioning members are configured to expand radially outward to position at least one of the injection ports in apposition with a wall of a blood vessel of a patient.

This application claims the benefit of U.S. Provisional Patent Application Ser. No. 63/476,974, filed Dec. 23, 2022, the entire content of which is incorporated herein by reference.

### TECHNICAL FIELD

The present technology is related to catheters.

### BACKGROUND

Catheters have been proposed for use with various medical procedures. For example, a catheter can be configured to deliver neuromodulation therapy to a target tissue site to modify the activity of nerves at or near the target tissue site. The nerves can be, for example, sympathetic nerves. The sympathetic nervous system (SNS) is a primarily involuntary bodily control system typically associated with stress responses. Chronic over-activation of the SNS is a maladaptive response that can drive the progression of many disease states. For example, excessive activation of the renal SNS has been identified experimentally and in humans as a likely contributor to the complex pathophysiology of arrhythmias, hypertension, states of volume overload (e.g., heart failure), and progressive renal disease.

### SUMMARY

The present disclosure describes a catheter including one or more noninflatable positioning members and one or more injection tubes configured to be positioned within a blood vessel of a patient to deliver therapy to the patient. The therapy can include, for example, neuromodulation therapy, such as renal denervation therapy. The one or more injection tubes each define at least one injection port configured to deliver a therapeutic agent, such as a chemical used for ablation of nerves, to a vessel wall or other tissue of the patient.

The one or more noninflatable positioning members are configured to expand radially outward (e.g., relative to a central longitudinal axis of the catheter) within the blood vessel to, for example, position the one or more injection ports tubes in apposition with a wall of the blood vessel to facilitate delivery of a therapeutic agent via the respective injection ports to tissue of the patient. For example, the one or more noninflatable positioning members can be configured to position the one or more injection ports to facilitate delivery of the therapeutic agent from into adventitial tissue or perivasculature tissue of a patient, such as renal perivasculature tissue, from a location within the blood vessel.

Various example configurations of the one or more noninflatable positioning members are described herein. For example, in some examples, a catheter includes two or more injection tubes, and the one or more noninflatable positioning members include at least one loop-shaped member radially inwards of the two or more injection tubes. As another example, the one or more noninflatable positioning members can define an expandable frame or a spiral, helical, or sinusoidal structure. Other configurations of noninflatable positioning members are described in further detail herein.

In some examples, the disclosure describes a catheter comprising: a catheter body defining a central longitudinal axis; one or more noninflatable positioning members connected to the catheter body; and one or more injection tubes disposed on an outer surface of the one or more noninflatable positioning member, each injection tube of the one or more injection tubes defining an injection tube lumen and one or more injection ports in fluid communication with the respective injection tube lumen, wherein the one or more noninflatable positioning members are configured to expand radially outward to position at least one of the one or more injection ports in apposition with a wall of a blood vessel of a patient.

In some examples, the disclosure describes a method comprising: navigating a catheter through the vasculature of a patient to a target treatment site, wherein the catheter comprises: a catheter body; one or more noninflatable positioning members connected to the catheter body; and one or more injection tubes disposed on an outer surface of the one or more noninflatable positioning members, each of the injection tubes of the one or more injection tubes defining an injection tube lumen and one or more injection ports in fluid communication with the respective injection tube lumen; deploying the one or more noninflatable positioning members to an expanded configuration to position at least one of the one or more injection ports in apposition with a wall of a blood vessel of a patient; and delivering therapy to the target treatment site via the one or more injection ports of the one or more injection tubes.

In some examples, the disclosure describes a catheter comprising: a catheter body defining a catheter lumen and a central longitudinal axis; an inner member disposed within the catheter lumen; one or more noninflatable positioning members; and one or more injection tubes connected to the one or more noninflatable positioning members, each injection tube of the one or more injection tubes defining an injection tube lumen and one or more injection ports in fluid communication with the respective injection tube lumen, wherein in response to movement of the inner member relative to the catheter body, the one or more noninflatable positioning members and the one or more injection tubes are configured to move radially outwards away from the central longitudinal axis.

Further disclosed herein is a catheter that comprises a catheter body, one or more noninflatable positioning members configured to deploy from a collapsed configuration to an expanded configuration, and one or more injection tubes connected to the one or more noninflatable positioning members, wherein each of the injection tubes defines an injection tube lumen and one or more injection ports in fluid communication with the respective injection tube lumen, wherein the one or more noninflatable positioning members are configured to expand radially outward to position at least one of the injection ports in apposition with a wall of a blood vessel of a patient.

The details of one or more aspects of the disclosure are set forth in the accompanying drawings and the description below. Other features, objects, and advantages of the techniques described in this disclosure will be apparent from the description and drawings, and from the claims.

### BRIEF DESCRIPTION OF THE DRAWINGS

FIG. 1 is a schematic illustration of an example medical system including a medical system.

FIG. 2 is a schematic illustration of the medical system of FIG. 1 within a blood vessel of a patient.

FIG. 3A is a conceptual diagram illustrating an example catheter system including injection tubes and noninflatable positioning members in a collapsed configuration.

FIG. 3B is a conceptual diagram illustrating the example catheter system of FIG. 3A in an expanded configuration.

FIG. 4A is a conceptual diagram illustrating another example catheter system, including a loop-shaped noninflatable positioning member, in a collapsed configuration.

FIG. 4B is a conceptual diagram illustrating the example catheter system of FIG. 4A in an expanded configuration.

FIG. 5 is a conceptual diagram illustrating another example catheter system including a plurality of shape memory arms connected to respective injection tubes via bands.

FIG. 6A is a conceptual diagram illustrating another example catheter system including a single loop-shaped noninflatable positioning member.

FIG. 6B is a conceptual diagram illustrating the example catheter system of FIG. 6A including multiple loop-shaped noninflatable positioning members.

FIG. 7 is a conceptual diagram illustrating another example catheter system including at least one shape memory arm connected to a distal portion of respective injection tubes.

FIG. 8A is a conceptual diagram illustrating a side view of another example catheter system including a noninflatable positioning member including a support frame in a partially collapsed configuration.

FIG. 8B is a conceptual diagram illustrating the example catheter system of FIG. 8A in an expanded configuration.

FIG. 9A is a conceptual diagram illustrating another example catheter system with a noninflatable positioning member in a collapsed configuration.

FIG. 9B is a conceptual diagram illustrating another example catheter system of FIG. 9A in an expanded configuration.

FIG. 10 is a conceptual diagram illustrating another example catheter system including noninflatable positioning members defining at least one spiral, helical, or sinusoidal structure.

FIG. 11 is a conceptual diagram illustrating another example catheter system including noninflatable positioning members defining a single a spiral, helical, or sinusoidal structure.

FIG. 12 is a flow diagram illustrating an example method of delivering therapy to a patient using a catheter comprising noninflatable positioning members.

### DETAILED DESCRIPTION

The disclosure describes devices, systems, and methods for neuromodulation, such as renal neuromodulation, using a therapeutic agent, such as an ablative chemical agent. Although the following examples will be described primarily with respect to renal neuromodulation, a person having ordinary skill in the art will understand that the devices, systems, and methods described herein may be used for neuromodulation at any suitable intravascular location within a body of a patient.

Conditions such as arrhythmias, hypertension, states of volume overload (e.g., heart failure), and progressive renal disease due to excessive activation of the renal sympathetic nervous system (SNS), may be mitigated by modulating the activity of overactive nerves (neuromodulating), for example, denervating, or reducing the activity of the overactive nerves. Sympathetic nerves of the kidneys terminate in the renal blood vessels, the juxtaglomerular apparatus, and the renal tubules, among other structures. Renal neuromodulation, such as renal denervation, may be accomplished using one or more of a variety of treatment modalities, including radio frequency (RF) energy, microwave energy, ultrasound energy, a therapeutic agent, or the like. When using a therapeutic agent, a neuromodulation catheter may be delivered to a renal vessel, such as a renal artery or renal vein, of a patient. The neuromodulation catheter may include at least one port through which the therapeutic agent is delivered. The therapeutic agent is selected to modulate activity of one or more renal nerves adjacent to the renal vessel in which the neuromodulation catheter is positioned. For example, the therapeutic agent may be a neurotoxic chemical selected to chemically ablate the one or more renal nerves near the renal vessel.

In the case of chemical denervation, a medical system may be configured to pressurize a therapeutic agent fluid (e.g., a chemical agent and/or other therapy fluid) to cause delivery of the therapy agent to a target treatment site via a catheter. In some cases, tissue ablation can be accomplished using relatively high pressure chemical ablation. For example, a neuromodulation procedure can involve a high pressure introduction of a therapeutic agent into a wall of a blood vessel for neuromodulation of nearby nerves (e.g., sympathetic renal nerves in the case of a renal denervation procedure). In some cases, the therapeutic agent can be delivered without needles. For example, a therapeutic agent can be delivered at a relatively high pressure through one or more injection ports defined by one or more injection tubes positioned in a blood vessel, where the pressure is high enough to force the therapeutic agent into and/or through a blood vessel wall. The therapeutic agent may then diffuse around the target treatment site (e.g., nerves surrounding the blood vessel). Example pressures include, for example, 5 Megapascal (MPa) to 21 MPa (e.g., about 750 pounds per square inch (psi) to about 3046 psi), such as 10 MPa (or about 1450 psi).

The present disclosure describes example catheters that includes one or more injection tubes configured to be positioned by one or more noninflatable positioning members, e.g., within a blood vessel of a patient, the one or more injection tubes each defining one or more injection ports configured to deliver a therapeutic agent to a target tissue site of the patient to achieve a therapeutic outcome. The one or more noninflatable positioning members, which can also be referred to as noninflatable expandable members, are configured to deploy from a relatively low profile configuration to a radially expanded configuration to position at least one of the one or more injection ports in apposition with a wall of a blood vessel of a patient. In some examples, the catheter is needleless such that the chemical is delivered to target tissue of the patient via relatively high pressure needleless injection. The medical systems, as well as systems including the medical systems and methods of using the medical system, can be used for any suitable medical procedures, such as neuromodulation (e.g., denervation), that include delivering a therapy (e.g., chemical agents) to a target tissue site.

The one or more noninflatable positioning members are configured to expand radially outwards, e.g., relatively a central longitudinal axis of a catheter body, to bring one or more injection tubes each defining one or more injection ports into apposition with a vessel wall and hold the injection ports against the vessel wall during high-pressure injection of the chemical. For example, at least one injection tube can be mechanically connected to a positioning member, e.g., via a mechanical band, adhesive, welding, or the like, such that as the positioning member expands radially outward, the injection tube moves with the positioning member. Thus, in some examples, at least a portion or all of at least one of the one or more injection tubes is flexible, e.g., more flexible than the one or more noninflatable positioning members. In some examples, at least one noninflatable positioning member is positioned in an injection tube. In some examples, a noninflatable positioning member also defines an injection tube, but the catheter includes at least one other injection tube that is separate from a noninflatable positioning member.

In examples in which a catheter includes multiple injection tubes, the injection ports of different injection tubes can be aligned or offset longitudinally (e.g., in a direction along a longitudinal axis of the catheter). In addition, the injection ports can be aligned circumferentially or circumferentially offset. The longitudinally offset injection ports can enable delivery of a therapeutic agent at multiple longitudinal locations along the vessel. The longitudinally aligned injection ports can help facilitate 360 degree delivery of the therapeutic agent around the vessel. The one or more positioning members are noninflatable in that, for example, they are configured to expand radially outward without the use of an inflation fluid (e.g., saline). In contrast to a balloon, the one or more noninflatable positioning members are configured to enable blood to continue flowing through the vessel, e.g., around and distally past the one or more noninflatable positioning members, throughout a neuromodulation procedure.

In some examples, the noninflatable positioning members are configured to self-expand. In addition to, or instead of self-expanding, the noninflatable positioning member can be configured to expand when a control member (e.g., a tube or a pull wire) connected to the noninflatable positioning member is withdrawn in a proximal direction or advanced in a distal direction. The noninflatable positioning member can have a shape that enables it to collapse into a relatively low profile configuration for delivery through vasculature to the target tissue site. For example, the noninflatable positioning member, when expanded, can define a sinusoidal wave or a tilted elliptical shape hoop to enable the hoop to collapse into a lower profile delivery configuration.

In some examples, the catheter includes two injection tubes spaced about 180 degrees apart. Other injection tube configurations can also be used in other examples. In some examples, the one or more noninflatable positioning members and/or another part of the catheter are configured to orient the injection ports of the injection tubes in a desired orientation, which can include, for example, a preferred direction of therapeutic agent delivery, e.g., in a direction away from renal veins when the catheter is positioned in a renal artery.

In some examples, the one or more injection tubes include a first injection tube and a second injection tube, and the first injection tube is configured to remain stationary while at least one noninflatable positioning member is configured to move the second injection tube away from the first injection tube when at least one noninflatable positioning member expands radially outward. In some examples, the at least one noninflatable positioning member defines a spiral, helical, or sinusoidal structure in the expanded state.

In some examples, the one or more injection tubes and one or more noninflatable positioning members are configured to collapse to a lower profile state by tilting towards a central longitudinal axis of the catheter body when the control member is pushed distally. The one or more noninflatable positioning members may be configured to expand radially outward when the control member is pulled proximally. In some examples, the one or more injections tubes and the one or more noninflatable positioning members are configured to collapse to the lower profile state or expand radially outwards when the control member is pulled proximally or pushed distally, respectively. The one or more noninflatable position members may be configured to self-expand. The one or more noninflatable positioning members may include nitinol.

The injection tubes can be fluidically coupled to a source of therapeutic agent using any suitable technique. In examples, the catheter body defines a catheter lumen and includes a fluid delivery tube disposed within the catheter lumen. Each of the injection tubes of the one or more injection tubes may include a proximal portion disposed within the catheter lumen and in fluid communication with the fluid delivery tube. The one or more injection tubes may include a plurality of injection tubes, and each injection tube of the plurality of injection tubes exits the catheter lumen and extends along an outer surface of the catheter body proximal to a proximal end of the one or more noninflatable positioning members.

In some examples, the catheter body defines a catheter lumen configured to receive a control member that is mechanically connected to the one or more noninflatable positioning members. The control member may include a guidewire tube defining a guidewire lumen configured to receive a guidewire. In an example, the control member includes one or more pull wires disposed within the catheter lumen.

While blood vessels are primarily referred to throughout the disclosure, the devices, systems, and techniques described herein are also applicable to other target tissue sites. Although the present technology is herein described in many instances with reference to renal nerves and vessels, the present technology also has application to neuromodulation at other anatomical sites (e.g., spinal neuromodulation, cardiac neuromodulation, brain neuromodulation, sacral neuromodulation, urinary neuromodulation, and/or neuromodulation techniques directed to other portions of a body) and their associated nerves and that such devices and systems can be configured (e.g., have suitable shape and dimensions) for such sites. For example, a catheter may be configured to deliver energy with a portion of the catheter defining injection ports, longitudinally aligned along injection tubes, that are positioned with a particular anatomical lumen or a particular tissue (e.g., a renal artery, external iliac artery, internal iliac artery, internal pudendal artery, celiac artery, mesenteric artery, superior mesenteric artery, inferior mesenteric artery, hepatic artery, splenic artery, gastric artery, left gastric artery, pancreatic artery, uterine artery, ovarian artery, testicular artery, and/or their associated arterial branches, accessories, veins, and/or other hollow anatomical structures).

As used herein, the terms “distal” and proximal” define a position or direction with respect to the treating clinician or clinician's control device (e.g., a handle assembly). “Distal” or “distally” can refer to a position distant from or in a direction away from the clinician or clinician's control device. “Proximal” and “proximally” can refer to a position near or in a direction toward the clinician or clinician's control device.

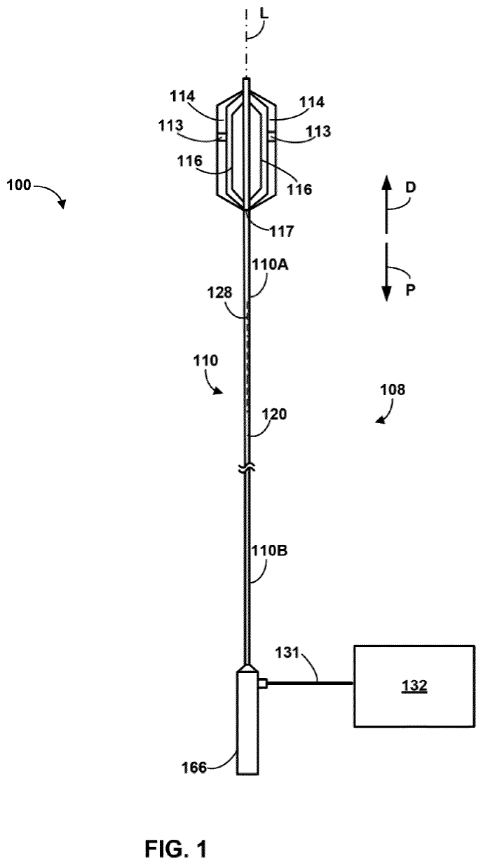

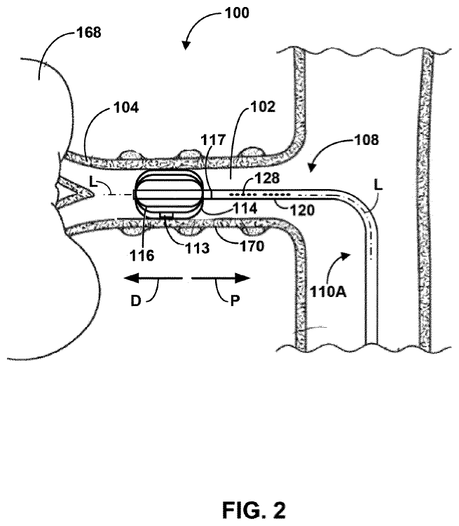

FIG. 1 is a schematic perspective view of an example medical system 100 including a catheter system 108 that includes an elongated body 110 defining a proximal body portion 110A and a distal body portion 110B configured to be positioned (e.g., by a clinician) within a blood vessel 102 (shown in FIG. 2) of a patient, one or more injection tubes 114, and one or more noninflatable positioning members 116. Noninflatable positioning members 116 are configured to expand radially outward to, for example, assist in positioning injection ports in apposition with vessel wall 104, assist in maintaining elongate body 110 and/or centering elongate body 110 within blood vessel 102, assist in displacing and/or maintaining a displacement between elongate body 110 and vessel wall 104, or any combination thereof.

For example, expansion of one or more noninflatable positioning members 116 radially outwards can also cause one or more injection tubes 114 to also move radially outwards to help position injection ports 113 in apposition with tissue of a patient.

Medical system 100 further includes a fluid delivery system 132, a fluid delivery tube 131 in fluid communication with injection tubes 114 via therapy fluid flow path 128, and handle 166.

FIG. 2 is a schematic illustration of a portion of medical system 100 within blood vessel 102 of the patient, blood vessel 102 having vessel wall 104. In the examples of FIG. 2, blood vessel 102 is a renal artery and vessel wall 104 is a renal artery wall. However, in other examples, medical system 100 is configured to deliver treatments to other blood vessels, anatomical lumens, and/or other tissues, such as an external iliac artery, internal iliac artery, internal pudendal artery, celiac artery, mesenteric artery, superior mesenteric artery, inferior mesenteric artery, hepatic artery, splenic artery, gastric artery, left gastric artery, pancreatic artery, uterine artery, ovarian artery, testicular artery, and/or their associated arterial branches, accessories, veins, and/or other hollow anatomical structures of a patient.

In examples, medical system 100 is configured to provide neuromodulation to the patient. Neuromodulation (e.g., renal neuromodulation) is the partial or complete incapacitation or other effective disruption of nerves (e.g., nerves terminating in the kidneys or in structures closely associated with the kidneys). Neuromodulation can include inhibiting, reducing, and/or blocking neural communication along neural fibers (e.g., efferent and/or afferent neural fibers). Such incapacitation can be long-term (e.g., permanent or for periods of months, years, or decades) or short-term (e.g., for periods of minutes, hours, days, or weeks). Neuromodulation may include renal denervation by high-pressure needless injection of a therapeutic agent (e.g., chemical) into renal arteries for ablation of sympathetic nerves. Neuromodulation may be expected to contribute to the systemic reduction of sympathetic tone or drive and/or to benefit at least some specific organs and/or other bodily structures innervated by sympathetic nerves. Accordingly, neuromodulation may be expected to be useful in treating clinical conditions associated with systemic sympathetic overactivity or hyperactivity, particularly conditions associated with central sympathetic overstimulation.

For example, renal neuromodulation may be expected to efficaciously treat hypertension, heart failure, acute myocardial infarction, metabolic syndrome, insulin resistance, diabetes, left ventricular hypertrophy, chronic and end stage renal disease, inappropriate fluid retention in heart failure, cardio-renal syndrome, polycystic kidney disease, polycystic ovary syndrome, osteoporosis, erectile dysfunction, and sudden death, among other conditions. The renal neuromodulation can be chemically induced or induced in another suitable manner or combination of manners (e.g., with one or more of electrically induced and thermally induced) at one or more suitable target sites during a treatment procedure. The target site can be within or otherwise proximate to a renal lumen (e.g., a renal artery, a ureter, a renal pelvis, a major renal calyx, a minor renal calyx, or another suitable structure), and the treated tissue can include tissue at least proximate to a wall of the renal lumen (e.g., proximate to vessel wall 104 of blood vessel 102). For example, with regard to a renal artery, a treatment procedure can include modulating nerves in the renal plexus, which lay intimately within or adjacent to the adventitia of the renal artery.

For ease of description, the following discussion will be primarily focused on delivering a therapeutic agent, such as a neurotoxic chemical or another chemical agent, to the target site.

Each of the injection tubes 114 defines at least one injection tube lumen (not shown) and one or more injection ports 113 in fluid communication with the respective injection tube lumen. Catheter system 108 is configured to deliver a therapeutic agent (e.g., a fluid) through the injection tubes 114 to the injection ports 113. For example, as part of a neuromodulation procedure, catheter system 108 can be used to deliver a therapeutic agent via one or more injection ports 113 to vessel wall 104 and/or other tissues associated with blood vessel 102. Once at a target site, the one or more noninflatable positioning members 116 can be expanded to cause the one or more injection tubes 114 to move radially outwards to position injection ports 113 in apposition with the vessel wall to facilitate delivery of a therapeutic agent therapy via injection ports 113 to tissue. For example, the injection ports 113 may be configured to deliver a therapeutic agent from an intravascular location to ablate tissue in adventitial or perivascular tissue or the like. Injection ports 113 may be, for example, a needleless injection nozzle and/or another component configured to deliver the therapy fluid to the target site. Injection ports 113 may be, for example, openings (e.g., of any shape, including substantially circular, rectangular, and so on) in the tubes, which can but need not be reinforced. Further, injection ports may be distributed equidistant or sporadically spaced longitudinally along injection tubes 114.

Injection tubes 114 are configured to withstand relatively high-pressure fluid delivery. When noninflatable positioning members 116 are in the expanded configuration, positioning members 116 may be configured to provide radial force against vessel wall 104 such that positioning members 116 resist the force during delivery the therapeutic agent to the target site, and help injection ports 113 maintain sufficient apposition to vessel wall 104.

In examples, injection ports 113 of the injection tubes 114 are configured to deliver the therapeutic agent to the adventitia and/or peri-adventitia, in which renal nerves are located. In examples, the therapy fluid is selected to neuromodulate (e.g., chemically ablate) nerve tissue of the renal plexus adjacent to a renal artery. The therapy fluid may include, for example, an alcohol, such as ethanol; distilled water; hypertonic saline; hypotonic saline; phenol; glycerol; lidocaine; bupivacaine; tetracaine; benzocaine; guanethidine; botulinum toxin; another appropriate neurotoxic fluid; or combinations thereof. In some examples, the therapy fluid may be heated or cooled to additionally or alternatively thermally ablate nerve tissue (e.g., nerve tissue of the renal plexus adjacent to renal artery).

In examples, noninflatable positioning members 116 are configured to expand radially outward to position at least one injection port 113 at least one of the one or more injection tubes 114 in apposition with vessel wall 104 of blood vessel 102 of patient. In contrast to a balloon that inflates within a blood vessel and occludes the blood vessel 102, noninflatable positioning members 116 are not expanded via an inflation fluid (e.g., saline). In addition, in some examples, noninflatable positioning members 116, when expanded, define gaps through which blood can flow. That is, in some examples, noninflatable positioning members 116, even if when expanded, do not occlude blood flow through blood vessel 102, thereby permitting blood to continuing flowing, e.g., to organs or the like, during a neuromodulation procedure and reducing a possibility of ischemia.

In addition, in contrast to some balloons, noninflatable positioning members 116 are configured to keep injection ports 113 exposed, even in a nonexpanded state. For example, when a balloon is deflated, e.g., during delivery to a target tissue site in a patient, the balloon may be folded and partially or even fully cover injection ports. Should the balloon not fully expand or otherwise un-occlude the injection ports during a medical procedure or even during testing of the device by a clinician prior to introducing the catheter into the patient, high-pressure delivery of therapeutic agent through the injection ports may adversely impact the integrity of the balloon.

In some examples, one or more (e.g., all) noninflatable positioning members 116 are configured to self-expand, e.g., are formed from any suitable material that, with limited or no external pressure exerted on the material, expands to a predetermined shape. In addition or instead, in some examples, one or more noninflatable positioning members 116 are configured to be expanded and contracted with the aid of a push or pull member. Further, noninflatable positioning members 116 are configured to collapse to a low-profile state. In the case of self-expanding members 116, for example, noninflatable positioning members 116 can occupy the low-profile configuration when external pressure is exerted on the members 116, for example, by a sheath, as discussed below. In some examples, noninflatable positioning members 116 comprise a shape memory material, such as nitinol (nickel titanium). In examples, noninflatable positioning members 116 may exhibit shape memory effect and superelasticity at varying temperatures. In other examples, noninflatable positioning members 116 comprise another material instead of or in addition to a shape memory material, such as, but not limited to, stainless steel or a polymer (e.g., polyamide).

Elongated body 110 defines a central longitudinal axis L and noninflatable positioning members 116 are configured to expand radially outwards relative to central longitudinal axis L (e.g., substantially perpendicular to longitudinal axis L). In examples, elongated body 110 is connected to (e.g., mechanically connected) noninflatable positioning members 116 and injection tubes 114. Injection tubes 114 may be positioned substantially external to noninflatable positioning members 116, such as on an outer surface of noninflatable positioning members 116. For example, injection tubes 114 are connected to an outer surface of the noninflatable positioning members 116 along at least the portion that is aligned with a distal portion 117 of the catheter lumen 120.

In examples, noninflatable positioning members 116 are more rigid than injection tubes 114, e.g., to enable noninflatable positioning members 116 to provide the force needed to hold injection ports 113 in apposition with a vessel wall during therapy delivery. Increasing the diameter (or other cross-sectional dimension) of noninflatable positioning members 116 may increase the overall rigidity of catheter system 108, which may interfere with navigation through vasculature to a target tissue site. Configuring catheter 108 to include one or more injection tubes 114 that are separate from the noninflatable positioning members 116 can help maintain a desired level of flexibility of catheter system 108, while maintaining a relatively large diameter (or other cross-sectional dimension) fluid delivery lumen. The larger diameter fluid delivery lumen(s) defined by injection tubes 114 can help configure catheter system 108 for relatively high pressure delivery of a therapeutic agent via injection ports 113, such that the therapeutic agent can penetrate vessel wall 104 without a needle. In contrast, with catheters in which the positioning members themselves (e.g., nitinol tubes) also define the injection port, increasing the diameter fluid delivery lumen may cause the overall rigidity of the catheter to be a hindrance to navigation through vasculature.

Catheter system 108 is configured to deliver a therapeutic agent into the injections tube lumen for delivery to tissue of the patient via the one or more injection ports 113 of the respective injection tube 114. For example, catheter system 108 can include a therapeutic agent delivery system 132 configured to provide the therapeutic agent to injection ports 113. Injection tubes 114 can be fluidically connected to therapy fluid delivery system 132 using any suitable technique. Catheter system 108 defines a therapeutic agent flow path 128 that fluidically couples therapy fluid delivery system 132 and injection tubes 114. In some examples, injection tubes 114 are fluidically connected to therapy fluid delivery system 132 via catheter lumen 120 of elongate body 110 or via a fluid flow path separate from catheter lumen 120. For example, each of the injection tubes 114 may include a proximal portion disposed within catheter lumen 120 and in fluid communication with a fluid delivery tube 131 that fluidically couples to therapeutic agent delivery system 132. In examples, catheter system 108 may be configured to commence, cease, adjust and/or substantially establish a flow rate of the therapeutic agent to adjust and/or establish increased pressure within the fluid delivery tube for high-pressure chemical ablation of vessel wall 104.

In some examples, therapy fluid delivery system 132 includes a therapy fluid container defining a therapy fluid reservoir (not shown) configured to hold a volume of the therapeutic agent. Therapy fluid delivery system 132 is configured to introduce the therapeutic agent from the therapy fluid reservoir to therapy fluid flow path 128 and into catheter lumen 120.

For example, therapy fluid delivery system 132 may be configured to pressurize the therapy fluid to discharge the therapy fluid from therapy fluid reservoir into therapy fluid flow path 128. Therapy fluid delivery system 132 may be configured such that the pressurization causes the therapy fluid within therapy fluid reservoir to substantially discharge from therapy fluid reservoir 132 into therapy fluid flow path 128. Therapy fluid flow path 128 may be configured to receive the pressurized therapy fluid from therapy fluid delivery system 132 and provide the pressurized therapy fluid to injection tubes 114. Therapy fluid delivery system 132 is be configured such that a clinician may cause pressurization of the therapy fluid to cause delivery to the target site. In examples, therapy fluid delivery system 132 is configured to pressurize the therapy fluid using a pressurization fluid such as pressurized carbon dioxide (CO₂).

Elongated body 110 defines a distal portion 110A (“distal body portion 110A”) and a proximal portion 110B (“proximal body portion 110B”). Noninflatable positioning members 116 and/or injection tubes 114 are positioned on distal portion 110A in the example shown in FIG. 1. Noninflatable positioning members 116 are configured to deploy from a relatively low profile delivery configuration to an expanded configuration to, for example, position injection ports 113 of injection tubes 114 against vessel wall 104 at the target treatment site. In some examples, catheter system 108 further includes an outer sheath configured to receive elongated body 110 and/or a guidewire configured to be received in a lumen of elongated body 110.

In some examples, system 100 includes the outer sheath is configured compress and hold noninflatable positioning members 116 in the low profile (e.g., linear) configuration In some of these examples, noninflatable positioning members 116 are configured to self-expand radially outwards in response to being deployed from the outer sheath. For example, the outer sheath can be pulled proximally away from distal portion 110A and/or elongated body 110 can be pushed distally out of the outer sheath. In addition to or instead of the outer sheath, in some examples, noninflatable positioning members 116 are configured to be deployed from the low profile configuration to the expanded configuration with the aid of a control member that is configured to directly or indirectly cause longitudinal movement of at least part of the noninflatable positioning members 116. For example, the control member can be a guidewire tube of system 100, which is configured to receive a guidewire and longitudinally move relative to at least part of elongated body 110. As another example, the control member can be a push and/or pull wire that is separate from a guidewire tube. The control member and the push/pull wire may be separately operable by the clinician, e.g., to control movement of catheter system 108 and expansion of noninflatable positioning members 116. For example, the clinician may advance catheter system 108 to the target tissue site via the control member and cause noninflatable positioning members 116 to radially expand via retraction or advancement of the push/pull wire. Thus, in some examples, the noninflatable positioning members are configured to expand in response to movement of a control member, such as a push and/or pull wire, a guidewire tube, or the like, to which the noninflatable positioning members 116 may be attached thereto.

In some examples, noninflatable positioning members 116 support injection tubes 114 and respective injection ports 113, such that, for example, expansion of noninflatable positioning members 116 decreases a displacement between the injection tubes 114 and, thus, injection ports 113 and vessel wall 104. For example, one or more injection tubes 114 may be supported by an outer surface of noninflatable positioning members 116. In some examples, elongate body 110 may be configured to support the noninflatable positioning members 116 and injection tubes 114 at a fixed longitudinal location (measured along longitudinal axis L) on elongate body 110 relative to noninflatable positioning members 116. In some examples, noninflatable positioning members 116 may take various configurations, such as sinusoidal, elliptical, flat, spiral, helical, a loop, expanded stents, or the like. Example configurations of noninflatable positioning members 116 are discussed in detail in the following figures. In some examples, a radiopaque material (not shown) may be attached to any of the noninflatable positioning members 116, injection tubes 114, and other parts of catheter system 10 to facilitate visualization of the position of injection ports 113 using medical imaging, e.g., fluoroscopy.

In some examples, medical system 100 (e.g., proximal body portion 110B) includes a handle portion 166, which is configured to remain outside vasculature of the patient when distal body portion 110A is within vasculature of the patient. Handle portion 166 may be configured to allow a clinician to navigate at least distal body portion 110A through the vasculature, allow pressurization of therapy agent, and/or enable other functions of medical system 100 which may assist in the delivery of a treatment (e.g., a neuromodulation) to patient. At least some portion of catheter system 108 (e.g., distal body portion 110A) may be substantially flexible, such that catheter system 108 may flex and/or bend enroute to positioning injection tubes 114 and respective injection ports 113 substantially at a target location within a blood vessel of a patient. Hence, although illustrated as substantially linear in FIG. 1, catheter system 108 (or portions thereof) may be configured to assume linear, curved, and/or curvilinear shapes. Correspondingly, longitudinal axis L (and/or portions thereof) defined by catheter system 108 may be linear, curved, and/or curvilinear.

FIG. 2 schematic illustrates a portion of medical system 100 (e.g., distal body portion 110A, injection port 113, injection tubes 114, and noninflatable positioning members 116) within blood vessel 102. Noninflatable positioning members 116 are configured to expand from a relatively low profile delivery configuration to an expanded configuration in which noninflatable positioning members 116 cause position injection ports 113 against, or proximate to, a target treatment site of vessel wall 104. That is, radially outward expansion of noninflatable positioning members 116 also causes one or more injection tubes 114 to expand radially outwards, which positions the respective injection ports 113 in contact with vessel wall 104. In examples, catheter system 108 may be configured to substantially center (e.g., center or nearly center to the extent permitted by vessel symmetry) distal body portion 110A within blood vessel 102 when noninflatable positioning members 116 expands to contact injection tubes 114 with vessel wall 104.

In examples, the radially outward force of the radially outward expansion of noninflatable positioning members 116 is strong enough to hold the injection ports 113 in apposition with vessel wall 104 during relatively high pressure therapeutic agent delivery, thereby enabling the therapeutic agent to pass through at least part of vessel wall 104 to target tissue, such as target nerves. Example pressures for the delivery of the therapeutic agent include, for example, for example, 5 Megapascal (MPa) to 21 MPa, such as 10 MPa (or about 1450 psi).

Injection ports 113 (e.g., via injection tubes 114) are configured to receive a therapeutic agent (e.g., via therapy fluid flow path 128) and deliver the therapeutic agent to, for example, ablate tissue of vessel wall 104 and/or tissues in proximity to vessel wall 104. Catheter system 108 may be configured may pressurize the therapy fluid (e.g., using therapy fluid delivery system 132 (FIG. 1)) to cause injection ports 113 to receive, and then deliver, the therapy fluid to a target treatment site of blood vessel 104. Injection ports 113 may transmit delivery of the therapy fluid to tissue of patient 106 to induce one or more desired effects (e.g., neuromodulation effects) on localized regions of blood vessel 102 and regions adjacent to blood vessel 102. For example, when blood vessel 102 defines a renal artery associated with a kidney 168, injection ports 113 may induce one or more desired neuromodulating effects to a portion of Renal Plexus (RP) 170 lying within or adjacent to the adventitia of the renal artery.

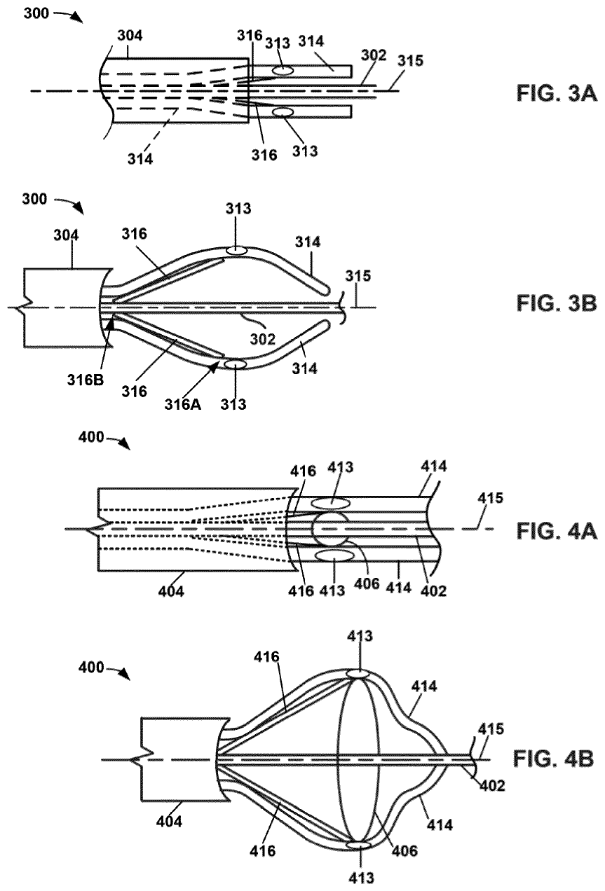

FIGS. 3A and 3B are conceptual diagrams illustrating an example catheter system 300 including injection tubes 314 and noninflatable positioning members 316. Catheter system 300, injection tubes 314, and noninflatable positioning members 316 are examples of catheter system 108, injection tubes 114, and noninflatable positioning members 116, respectively, of FIGS. 1 and 2. In FIG. 3A, noninflatable positioning members 116 are in a collapsed configuration within an outer sheath 304, and in FIG. 3B, noninflatable positioning members 316 are deployed from outer sheath 304 and in an expanded configuration.

Noninflatable positioning members 316 are configured to expand radially outward when deployed from outer sheath 304. In general, in examples described herein, deployment from an outer sheath can be accomplished using any suitable technique, such as, but not limited to, proximally withdrawing the outer sheath and/or pushing the positioning members or other catheter system component distally out of a distal opening of the outer sheath. When positioned in outer sheath 304, outer sheath 304 applies a compressive force to noninflatable positioning members 316, thereby holding noninflatable positioning members 316 in the collapsed configuration. When deployed from outer sheath 304, noninflatable positioning members 316 self-expand radially outwards to help bring injection tubes 314 into contact with a vessel wall.

Noninflatable positioning members 316 can be connected to one or more other components of catheter system 300 using any suitable technique and at any suitable location. For example, in some examples, proximal portions of noninflatable positioning members 316 are connected to a guidewire tube 302 of catheter system 300. Guidewire tube 302 is configured to receive a guidewire, e.g., to aid navigation of catheter system 300 to a target tissue site within vasculature of a patient. As another example, noninflatable positioning members 316 are not directly connected to guidewire tube 302, but rather, merely extend along part of guidewire tube 302.

In the example shown in FIG. 3A, noninflatable positioning members 316 include a plurality of shape memory arms, e.g., two, three, four or more arms. The shape memory arms are elongated shape memory structures and can have any suitable shape and form, e.g., can be flat wires, tubular wires, or the like. Noninflatable positioning members 316 may expand radially outward such that a distal portion of noninflatable positioning members 316 applies force to injection tubes 314 positioning injection tubes 314 against vessel wall 104 and injection ports 313 in contact, or proximate to, the target site of vessel wall 304. Noninflatable positioning members 316 may each contact one of injection tubes 314 at a single point (e.g., the distal portion) or at multiple points along the length of a surface of noninflatable positioning members 316.

In the example shown in FIGS. 3A and 3B, distal ends 316A of noninflatable positioning members 316 are mechanically connected (e.g., via an adhesive, an attachment device, welding, or the like) to a respective one or more injection tubes 314. For example, distal ends 316A of the noninflatable positioning members 316 can be connected to one injection tube 314. As an example, each distal end 316A of noninflatable positioning members 316 may be mechanically connected to one of injection tubes 313 (e.g., via a mechanical band, adhesive, welding, or the like) such that as the distal ends of noninflatable positioning member 316 moves radially away from guidewire tube 302, the injection tube 314 moves with the noninflatable positioning member 316. Noninflatable positioning members 316 may, e.g., be connected to injection tubes 314 proximate to injection ports 313 to support high-pressure therapeutic delivery and counter any pressure from delivering therapeutic agent to the target site of vessel wall 104. In some examples, distal ends 316A of noninflatable positioning members 316 may be midway between adjacent injection ports 313 of respective injection tubes 314.

In some examples, a proximal end 316B of each noninflatable positioning member 316 (e.g., proximal end 316B of each shape memory arm) is mechanically connected to guidewire tube 302 (e.g., via a mechanical band, adhesive, welding, or the like) such that as a distal end 316A of noninflatable positioning member 316 expands radially outward, proximal ends 316B of noninflatable positioning members 316 remain connected to guidewire tube 302. A length (measured along longitudinal axis 315) of each noninflatable positioning member 316 may be selected based on a diameter of vessel wall 104 or a range of diameters for which catheter system 300 is intended to be used (e.g., 3 millimeters (mm) to 8 mm). In some examples, noninflatable positioning members 316 have the same length. In other examples, two or more noninflatable positioning members 316 of catheter system 300 have different lengths to accommodate holding of different injection ports 113 at different longitudinal positions along vessel wall 104.

Distal ends 316A of the noninflatable positioning members 316 are not connected to guidewire tube 302, thereby enabling distal ends 316A to expand away from central longitudinal axis 315 to cause injection tubes 314 to also expand away from central longitudinal axis 315 and thereby bring injection tubes 314 into apposition with the vessel wall. In some examples, noninflatable positioning members 316 are radially inward of the injection tubes 314 so that noninflatable positioning members 316 can hold injection ports 313 in apposition with vessel wall 104 during delivery of the therapeutic agent to the target site.

Catheter system 300 can include any suitable number of injection tubes 314. As shown in FIG. 3A, in some examples, example catheter system 300 comprises two injection tubes 314, each including a respective injection port 313. In some examples, injection tubes 314 are disposed about 180 degrees (e.g., 180 degrees or nearly 180 degrees to the extent permitted by manufacturing tolerances) apart. The 180-degree separation can help facilitate delivery of a therapeutic agent 360 degrees around a vessel wall. In other examples, catheter system 300 comprises one injection tube or three, four, or more injection tubes 314, which can be evenly distributed around longitudinal axis 315 or can be unevenly distributed around longitudinal axis 315. An uneven distribution can help, for example, focus delivery of a therapeutic agent to only part of vessel wall 104 to target a particular nerve location or reduce delivery to non-target tissue sites.

Injection tubes 314 can define any suitable number of injection ports 313 to enable sufficient therapeutic agent to be delivered to a target tissue site, e.g., to achieve renal denervation. For example, a first injection tube 314 may comprise three injection ports 313 and a second injection tube 314 may comprise four injection ports 313. For example, a first injection tube 314 may comprise a single injection port 313 and a second injection tube 314 may comprise another single injection port 313. For example, a first injection tube 314 may comprise ten injection ports, longitudinally distributed along a length of injection tube 314, and a second injection tube 314 may comprise ten injection ports 313 longitudinally distributed along a length of the second injection tube 314.

Multiple injection ports 313 for each injection tube 314 may help increase the likelihood that at least one injection port 313 will be held in contact with a target site of vessel wall 104 when noninflatable positioning members 316 are expanded. Catheters described herein can be configured for use with a range of vessel sizes, e.g., 3 millimeters (mm) to 8 mm in diameter. Due to these different vessel sizes, when noninflatable positioning members 316 are expanded, different parts of injection tubes 314 may be in contact with vessel wall 104. Including a plurality of injection ports 313 on each injection tube 314 can, therefore, help accommodate different vessel 102 sizes.

FIGS. 4A and 4B are conceptual diagrams illustrating another example catheter system 400, which is similar to catheter system 300 of FIGS. 3A and 3B, but includes loop-shaped member 406 that is part of noninflatable positioning members 416. Loop-shaped member 406 may be radially inwards of the two or more injection tubes 414.

FIG. 4A illustrates an example collapsed configuration of noninflatable positioning members 416 and loop-shaped member 406. In examples, loop-shaped member 406 is configured to collapse to a lower profile state by at least tilting towards a central longitudinal axis 415 of guidewire tube 402 (which can also be a central longitudinal axis of the catheter) when noninflatable positioning members 416 and injection tubes 414 are within a lumen of outer sheath 404. For example, loop-shaped member 406 is configured to collapse to the lower profile state by at least tilting, from the expanded configuration, towards central longitudinal axis 415 of guidewire tube 402.

As shown in FIG. 4B, loop-shaped member 406 is configured to expand radially outwards towards an expanded configuration and mechanically support injection tubes 414 against vessel wall 104. In some examples, loop-shaped member 406 and the other noninflatable positioning members 416 expand radially outwards into the expanded configuration upon being deployed from outer sheath 404. In some examples, loop-shaped member 406 and noninflatable positioning members 416 expand radially towards the expanded configuration upon guidewire tube 402 being pulled proximally, e.g., by a clinician. In some examples, a proximal force is applied to guidewire tube 402 to retain loop-shaped member 406 and noninflated positioning members 416 in the lower profile state. Loop-shaped member 406 and noninflatable positioning members 416 may expand radially towards the deployed (expanded) configuration upon guidewire tube 406 being advanced distally, e.g., by a clinician.

In some examples, loop-shaped member 406 may take on any suitable shape in the expanded configuration, e.g., approximately circular, elliptical, and so on, and can be referred to as ring-shaped in some examples. The loop defined by loop-shaped member 406 can be a closed loop or open loop (e.g., a partial ring shape). In some examples, loop-shaped member 406 is formed from a shape memory material, such as nitinol (nickel titanium), and is configured to self-expand into the deployed configuration shown in FIG. 4B in response to being deployed from outer sheath 304. In other examples, loop-shaped member 406 comprises another material instead of or in addition to a shape memory material, such as, but not limited to, stainless steel or a polymer, and is configured to expand into the deployed configuration shown in FIG. 4B in response to proximal (or distal) movement of guidewire tube 402. In some examples, noninflatable positioning members 416 comprise nitinol while loop-shaped member 406 comprises stainless steel or a polymer. In some examples, loop-shaped member 406 comprises a composite material comprising two or more materials including, but are not limited to, two or more different polymers or a combination of one or more polymers and one or more metals. The two or more materials may form a mixed braid defining loop-shaped member 406.

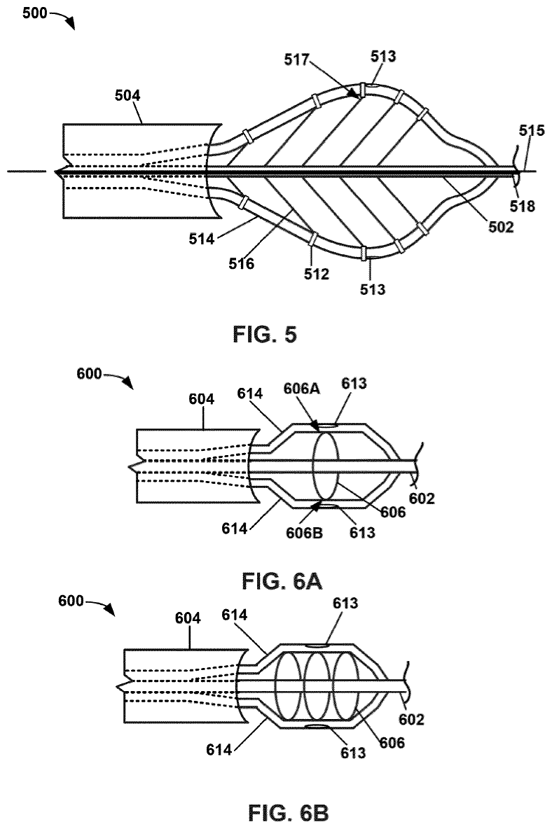

In some examples, loop-shaped member 406 is connected to the arm-like noninflatable positioning members 416 (e.g., via an adhesive, an attachment device, welding, or the like). In some examples, a band (e.g., band 512, FIG. 5) surrounds a respective injection tube and be mechanically connected to both loop-shaped member 406 and respective noninflatable positioning member 416.

In examples described with reference to at least FIGS. 3, 4, and 6-10, one or more noninflatable positioning members are configured to self-expand radially outwards and/or are configured to expand radially outwards with the aid of a control member. While the guidewire tube, described with respect to FIGS. 3, 4, 6-10 can act as control member, in some examples, a catheter can include a control member that is different from, separate from, or otherwise not a guidewire tube. For example, the clinician may separately manipulate the guidewire tube to navigate the example catheter within the vasculature of the patient and the control member to radially expand the one or more noninflatable positioning members, respectively.

FIG. 5 is a conceptual diagram illustrating another example catheter system 500, which is similar to catheter system 300 of FIGS. 3A and 3B, but includes a frame defined by a plurality of arms 516. Arms 516 are part of the noninflatable positioning member of catheter system 500. A plurality of arms 516 are connected to each injection tube 514 via bands 512 or using another suitable technique (e.g., an adhesive, welding, or the like). FIG. 5 illustrates an example expanded configuration of arms 516. Arms 516 are configured to expand radially outwards from a relatively low profile configuration to the expanded configuration and mechanically support positioning of injection tubes 514 against vessel wall 104. In some examples, arms 516 are formed from a shape memory material and expand radially outwards to define an expanded frame in response to being deployed from outer sheath 504. In some examples, arms 516 are not self-expanding and are configured to expand radially outwards in response to control member 502 (or a separate control member) being pulled proximally or pushed distally, e.g., by a clinician. In these examples, catheter system 500 does not include outer sheath 504.

Arms 516 are positioned radially inwards of the two or more injection tubes 514, such that the radially outward force of the frame defined by arms 516 helps hold injection ports 513 in apposition to a vessel wall. Arms 516 can have any suitable configuration. For example, the length of each of the arms 516 may depend on a size of target treatment site and the distance from vessel wall 104 to a center longitudinal axis 515 of guidewire tube 505.

Further, there may be any number of arms 516 connected to each injection tube 514, and each injection tube 514 can be connected to the same number of arms or a different number of arms. For example, there can be ten arms 516 mechanically connected to a first injection tube 514 and another ten arms 516 connected to a second injection tube 514, and the one or more of arms 516 connected to either first or second injection tube 514 may have varying dimensions.

Arm 516 are mechanically connected to injection tubes 514 using any suitable technique, e.g., via an adhesive, an attachment device, welding, or the like. In some examples, each arm 516 is mechanically connected to one of two injection tubes 514 via a band 512. Each band 512 may encircle a distal portion 517 of each respective arm 516 and the respective injection tube 514. Bands 512 may be of a circumference such that bands 512 are in fixed positions relative to injection tubes 514 or such that bands 512 can freely traverse longitudinally along injection tubes 514. In some examples, a clinician can control noninflatable positioning members 516 expanding to the expanded configuration or collapsing to a collapsed configuration using control member 502. For example, as a clinician pushes control member 502 distally, noninflatable positioning members 516 expand to an expanded configuration, and bands 512 may traverse along injection tubes 514 until the noninflatable positioning members 516 are in the expanded configuration. For example, the clinician pulls control member 502 proximally to collapse noninflatable positioning members 516 to the collapsed configuration. In some examples, a pull wire 518 may be separate from control member 502.

In some examples, a clinician can control noninflatable positioning members 516 expanding to the expanded configuration or collapsing to the collapsed configuration using pull wire 518. For example, as a clinician pulls pull wire 518 proximally, noninflatable positioning members 516 collapse to a collapsed configuration, and bands 512 traverse along injection tubes 514 until the noninflatable positioning members 516 are in the collapsed configuration. For example, the clinician may push pull wire 518 distally to expand noninflatable positioning members 518 to the expanded configuration. Arms 516 may be connected to one or both of control member 502 or pull wire 518, e.g., via an adhesive, welding, or the like.

FIGS. 6A and 6B are conceptual diagrams illustrating another example catheter system 500, which includes at least one loop-shaped noninflatable positioning member 606.

FIG. 6A illustrates catheter system 600 including a single loop-shaped noninflatable positioning member 606 and FIG. 6B illustrates catheter system 600 including a plurality of loop-shaped noninflatable positioning member 606 distributed along a longitudinal axis of the catheter.

Catheter system 600 is similar to catheter systems 400 of FIGS. 4A and 4B, but the noninflatable positioning member does not include arms. Loop-shaped noninflatable positioning member(s) 606 are radially inwards of and attached to injection tubes 614, each of injection tubes 614 defining at least one injection port 613. In the example shown in FIG. 6A, a first portion 606A of positioning member 606 and a second portion 606B of positioning member 606 are connected (e.g., via a mechanical band, adhesive, welding, or the like) to respective injection tubes 614. In some examples, each loop-shaped member 606 is connected to injection tubes 614 via a band (e.g., band 512) surrounding each loop-shaped noninflatable positioning member 606 and respective injection tube 614.

In some examples, loop-shaped noninflatable positioning members 606 are connected to any number of injection tubes 614 (e.g., three, four, or more). In examples, the circumference of each loop-shaped member 606 may have a range of values and each loop-shaped noninflatable positioning member 606 may have same or different circumferences (or other maximum cross-sectional dimension) relative to each other. Loop-shaped noninflatable positioning members 606 can have any suitable shape and form, e.g., can be elliptical, circular, oval, or the like. In examples, there may be any quantity of loop-shaped noninflatable positioning members 606 distributed longitudinally along guidewire tube 602 and placed in contact with injection tubes 614 at any location, e.g., proximate to injection ports 613 to counter any pressure from delivering therapeutic agent to a target treatment site of vessel wall 104. For example, a loop-shaped noninflatable positioning member 606 may be adjacent or directly under an injection port 613, or two loop-shaped members 606 may be proximate to, or on opposite sides of, injection port 613.

In some examples, loop-shaped noninflatable positioning members 606 are self-expanding and are configured to expand radially outward when deployed from outer sheath 604. When positioned in outer sheath 604, outer sheath 604 applies a compressive force to loop-shaped noninflatable positioning members 606, thereby holding loop-shaped noninflatable positioning members 606 in the collapsed configuration (not shown in FIG. 6A or 6B). When in the collapsed configuration, each loop-shaped noninflatable positioning member 606 may be in contact with guidewire tube 602. When deployed from outer sheath 604, loop-shaped noninflatable positioning members 606 self-expand radially outwards to help bring injection tubes 614 into contact with vessel wall 104.

In other examples, in addition to or instead of being self-expanding, loop-shaped noninflatable positioning members 606 are configured to expand radially outwards in response to proximal or distal movement of guidewire tube 602 or separate control member.

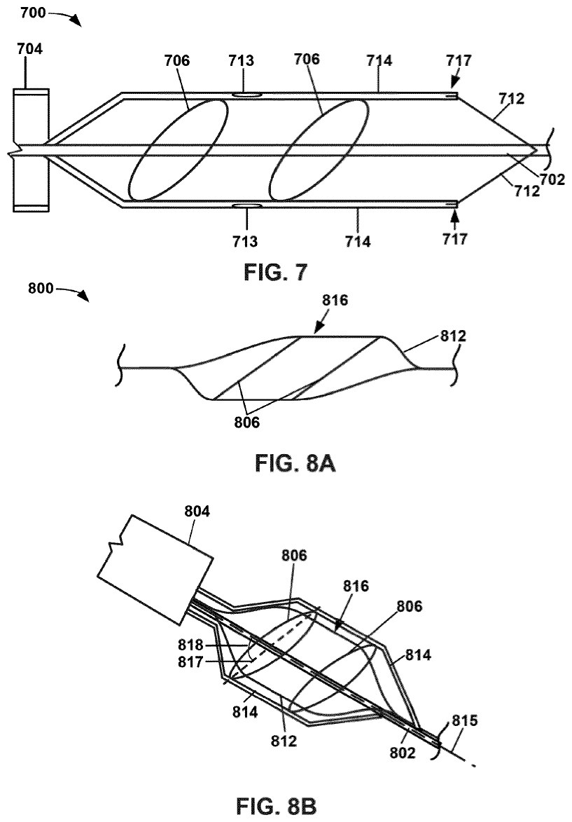

FIG. 7 is a conceptual diagram illustrating another example catheter system 700, which is similar to catheter systems 600 of FIGS. 6A and 6B, but includes at least one shape memory arm 712 connected to a distal portion 717 of respective injection tubes 714. Loop-shaped members 706 may be substantially similar to loop-shaped members 606. In some examples, shape memory arms 712 are mechanically connected to (e.g., via an adhesive, an attachment device, welding, or the like) injection tubes 704 at distal portion 717. Loop-shaped members 706 are mechanically connected to (e.g., via an adhesive, an attachment device, welding, or the like) to injection tubes 714. In some examples, shape memory arms 712 are configured to expand radially outwards and mechanically support loop-shaped members 706 in positioning injection tubes 714 against vessel wall 104. For example, shape memory arms 712 can mechanically support distal portion 717 of injection tubes 714 while loop-shaped members 706 support proximal portions (relative to distal portion 717) of injection tubes 714 in positioning injection tubes 714 against vessel wall 104.

In some examples, shape memory arms 712 are self-expanding and are configured to expand radially outward when deployed from outer sheath 604. In other examples, in addition to or instead of being self-expanding, shape memory arms 712 are configured to expand radially outwards in response to proximal or distal movement of guidewire tube 702 or separate control member. In some examples, shape memory arms 712 provide additional (in addition to loop-shaped members 706) radially outward force to injection tubes 714. For example, shape memory arms 712 are configured to apply a radially outward force to distal portion 717 of injection tubes 714 so that, as guidewire tube 702 is pulled proximally, loop-shaped members have less resistance from injection tubes 714 when loop-shaped members 706 expand to the expanded configuration. In some examples, when the clinician collapses loop-shaped rings 706 and injection tubes 714 into outer sheath 704, shape memory arms 712 help balance a proximal force applied by the clinician along guidewire tube 702 across injection tubes 714 and loop-shaped members 706, e.g., to facilitate an unbiased collapse of system 700.

FIGS. 8A and 8B are conceptual diagrams illustrating another example catheter system 800, which includes a noninflatable positioning member that includes a support frame 816. FIG. 8A illustrates catheter system 800 including support frame 816 in an expanded configuration and FIG. 8B illustrates catheter system 800 including support frame 816 in an expanded configuration.

Support frame 816 includes an outer frame portion 812 and loop-shaped members 806 radially inwards of outer frame portion 812. Outer frame portion 812 supports injection tubes 814 and also applies radially outward force against injection tubes 814. In some examples, outer frame portion 812 is configured to support loop-shaped members 806 in positioning injection tubes 814 against vessel wall 104. Loop-shaped members 806 are configured to help expand outer frame portion 812 radially outwards from a collapsed configuration, including the partially collapsed configuration of FIG. 8A, towards an expanded configuration of FIG. 8B and mechanically support in positioning injection tubes 814 against vessel wall 104. In some examples, loop-shaped member 806 and outer frame portion 812 self-expand radially outwards from the collapsed configuration towards the expanded configuration upon outer sheath 804 being pulled proximally and/or being distally advanced out of a distal opening of outer sheath 804. In some examples, loop-shaped member 806 and outer frame portion 812 expand radially towards the expanded configuration in response to guidewire tube 802 being pulled proximally, e.g., by a clinician. In some examples, guidewire tube 802 is connected to outer frame portion 812 via an adhesive, an attachment device, welding, or the like.

In some examples, loop-shaped members 806 facilitate outer frame portion 812 fully or nearly fully expanding to the expanded configuration. For example, in a fully expanded configuration a plane 817 (only shown as one dimensional for ease of illustration) defined by each loop-shaped member 806 may be separated from central longitudinal axis 815 of guidewire tube 802 by an angle 818. Angle 818 may be up to about 90 degrees. Angling loop-shaped members 806 at angle 818 (e.g., 90 degrees or less than 90 degrees) may facilitate collapse of support frame 816, e.g., by reducing an amount of force required to collapse support frame 816 and/or by allowing loop-shaped members 806 to collapse over a wider area of support frame 816.

In examples, loop-shaped member 806 defines any suitable loop-shape in the expanded configuration, e.g., approximately circular, elliptical, and so on, which can be a closed loop or an open loop. In some examples, loop-shaped member 806 and outer frame portion 812 comprises a shape memory material, such as nitinol (nickel titanium) and are configured to self-expand, e.g., in response to being deployed from outer sheath 804. In other examples, loop-shaped member 806 and outer frame portion 812 comprise another material instead of or in addition to a shape memory material, such as, but not limited to, stainless steel or a polymer. In some examples, outer frame portion 812 comprises nitinol while loop-shaped member 806 comprises stainless steel or a polymer. In some examples, loop-shaped member 806 is connected to outer frame portion 812 (e.g., via an adhesive, an attachment device, welding, or the like). In some examples, a band (e.g., band 512, FIG. 5) surrounds a respective loop-shaped member 806 and be mechanically connected to both outer frame portion 812 and injection tube 814.

As discussed above, in some examples, a catheter system includes a first noninflatable positioning member connected to a first injection tube and configured to remain relatively stationary (e.g., in a linear configuration) while a second noninflatable positioning member connected to a second injection tube expands away from the first noninflatable positioning member (e.g., to define a non-linear configuration). Radially outward expansion of the second noninflatable positioning member moves the second injection tube away from the first injection tube. In some examples, the first injection tube is not connected to a positioning member and may remain relatively stationary while a noninflatable positioning member expands to move the second injection tube away from the first injection tube.

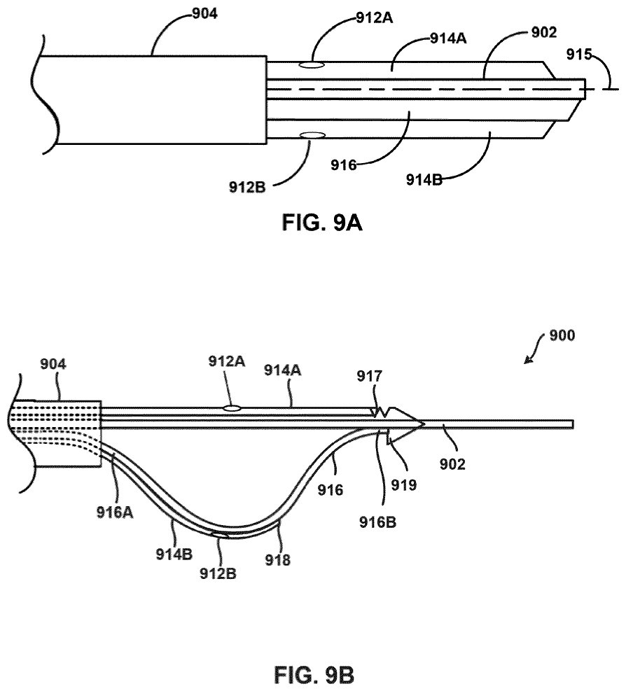

FIGS. 9A and 9B are conceptual diagrams illustrating an example catheter system 900 that includes a first injection tube and a second injection tube configured to move away from the first injection tube, e.g., while the first injection tube remains relatively stationary and/or in a linear configuration. Catheter system 900 includes a guidewire tube 902, an outer sheath 904, a first injection tube 914A and a second injection tube 914B, each with respective injection ports 912A and 912B, and a noninflatable positioning member 916.

FIG. 9A illustrates an example collapsed configuration of noninflatable positioning member 916. In examples, noninflatable positioning member 916 is configured to collapse to a lower profile state by at least moving, from the expanded configuration shown in FIG. 9B, towards a central longitudinal axis 915 of guidewire tube 902 (which can also be a central longitudinal axis of the catheter) when noninflatable positioning member 916 and injection tubes 914 are within a lumen of outer sheath 904.

As shown in FIG. 9B, noninflatable positioning member 916 is configured to self-expand radially outward to an expanded configuration moving second injection tube 914B away from first injection tube 914A that remains substantially linear relative to second injection tube 914B. In the example shown in FIG. 9B, noninflatable positioning member 916 is connected to second injection tube 914B, and is unconnected to first injection tube 914A.

For example, when noninflatable positioning member 916 expands from a relatively low profile collapsed configuration to an expanded configuration, noninflatable positioning member 916 expands radially outward to position second injection tube 914B against vessel wall 104 while first injection tube 914A remains relatively stationary and/or linear, e.g., against a vessel wall.

For example, first injection tube 914A is connected to guidewire tube 902 which does not move with noninflatable positioning member 916. First injection tube 914A remaining relatively stationary and/or linear may increase the ease and predictability of positioning injection port 912A in apposition with the target treatment site. For example, injection port 912A along injection tube 914A will remain in contact with vessel wall 104 even when noninflatable positioning member 916 is expanded. In contrast, whether injection port 912B of injection tube 914B will be in direct contact with vessel wall 104 may depend on the size of the blood vessel and the location of the apex of noninflatable positioning member 916. In examples, the force of noninflatable positioning member 916 extending radially outward positioning injection tube 914B against vessel wall 104 will also position first injection tube 914A against another portion of vessel wall 104. Injection tubes 914A, 914B can include any suitable number of injection ports.

In some examples, one or more of first injection tube 914A, second injection tube 914B, and noninflatable positioning member 916 taper at distal portion 917, distal portion 918, and distal portion 919, respectively. Tapering of distal positions 917, 918, and 919 may facilitate navigation of catheter system 900 through blood vessel 102.

In some examples, a proximal portion 916A and a distal portion 916B of noninflatable positioning member 916 are both permanently connected to (e.g., via an adhesive, an attachment device, welding, or the like) guidewire tube 902. In some examples, first injection tube 914A is permanently connected to (e.g., via an adhesive, an attachment device, welding, or the like) guidewire tube 902 along the longitudinal length of first injection tube 914A. To return noninflatable positioning member 916 and injection tubes 914 to a collapsed configuration, catheter system 900 can be pulled proximally and/or sheath 904 can be pushed distally until noninflatable positioning member 916 and injection tubes 914 are within the lumen of sheath 904.

In other examples of catheter system 900, either injection tube 914A or 914B may be eliminated. For example, in examples in which catheter system 900 includes injection tube 914A, but not injection tube 914B, noninflatable positioning member 916 may help hold injection port 912A defined by injection tube 914B in apposition with vessel wall 104 during relatively high pressure injection of a therapeutic agent through injection tube 914A and port 912A.

Noninflatable positioning members 916 as well as other noninflatable positioning members described herein can be formed from any suitable one or more materials. In some examples, noninflatable positioning members 916 comprises a braided shaft. The braided shaft may comprise any suitable material, e.g., polymer or nitinol braided wires. The braided shaft may increase, e.g., ease of navigation through blood vessel 102 or delivery of therapeutic agent to target treatment site by increasing pushability, steerability, high-torque maneuverability, burst-pressure resistance from delivery of therapeutic agent, kink resistance, and the like compared to a nonbraided configuration. In some examples, noninflatable positioning members 916 comprise a nitinol tube with a cross-sectional area of approximately 0.030 mm×0.035 mm (0.012 inches (in)×0.014 in), 0.025mm×0.030 mm (0.010 in×0.012 in), or the like. In some examples, noninflatable positioning members 916 comprise a polyimide tube and a nitinol strip (e.g., any suitable number of nitinol strips, including one, two, or more) and/or nitinol wires (e.g., any suitable number of nitinol wires, including one, two, or more).

Injection tubes 914A and 914B can define any suitable number of injection ports to enable sufficient therapeutic agent to be delivered to a target treatment site, e.g., to achieve renal denervation. In one example, injection tube 914A may comprise three injection ports 913 and injection tube 914B may comprise four injection ports 913. In another example, injection tube 914A may comprise a single injection port 913 and injection tube 914B may comprise another single injection port 913. In another example, injection tube 914A may comprise no injection ports 913 and injection tube 914B may comprise one injection port 913.

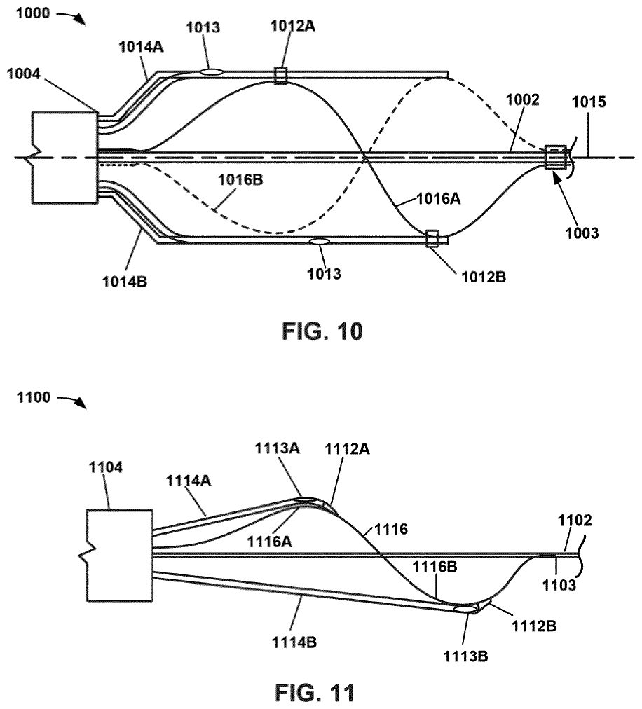

As discussed above, in some examples, a noninflatable positioning member is positioned radially inward of one or even all of the injection tubes to enable the positioning members to bias the injection tubes radially outwards, e.g., against a vessel wall. The noninflatable positioning member can have include one or more arms (e.g., as shown in FIGS. 3A-5), a loop-shaped configuration (e.g., as shown in FIGS. 4A, 4B, and 6A-8B), a frame-like structure (e.g., as shown in FIGS. 8A and 8B), or the like, as discussed above, or can include another suitable configuration. For example, as discussed with reference to FIG. 10, noninflatable positioning members can have a spiral, helical, or sinusoidal configuration and be connected to one or more injection tubes. The spiral, helical, or sinusoidal noninflatable positioning member are configured to expand radially outwards to help position one or more injection ports defined by the one or more injection tubes in apposition with vessel wall 104.

FIG. 10 is a conceptual diagram illustrating example catheter system 1000, which includes noninflatable positioning members 1016A and 1016B defining a spiral, helical, or sinusoidal structure in the expanded configuration. Catheter system 1000 includes a guidewire tube 1002, sheath 1004, injection tubes 1014A and 1014B, and noninflatable positioning members 1016A, 1016B. Rather than having one or more noninflatable positioning members per injection tube as is described with respect to some other examples, catheter system 1000 includes common noninflatable positioning members 1016A, 1016B for multiple injection tubes 1014A and 1014B. Noninflatable positioning members 1016A, 1016B that is configured to expand radially outwards from a collapsed configuration to the expanded configuration and mechanically support positioning of multiple injection tubes 1014A, 1014B against vessel wall 104.

Noninflatable positioning members 1016A, 1016B define a spiral, helical, or sinusoidal structures in the expanded configuration. In some examples, noninflatable positioning members 1016A, 1016B are arranged so that respective peaks of noninflatable positioning members 1016A, 1016B are offset from each other. For example, the spiral or helical structures can be rotated and/or shifted longitudinally with respect from one another. For example, noninflatable positioning members 1016A, 1016B may have substantially similar (e.g., the same but for manufacturing tolerances) configurations and noninflatable positioning member 1016B may be rotated about 15 degrees to about 180 degrees with respect to positioning member 1016A about center longitudinal axis 1015, such as 90 degrees to 180 degrees, and/or longitudinally displaced from noninflatable positioning member 1016A along longitudinal axis 1015. In other examples, noninflatable positioning members 1016A, 1016B can define other shapes, such as sinusoidal or other curvilinear shapes that facilitate positioning of injection ports of multiple injection tubes against vessel wall 104 during relatively high pressure delivery of therapeutic agent through the injection ports.

In some examples, there are two noninflatable positioning members 1016A, 1016B, only one positioning member 1016A or 1016B, or more than two noninflatable positioning members (e.g., three, four, or more). In some examples, noninflatable positioning members 1016A and B are each connected to respective injection tubes 1014A and 1014B at at least two locations 1012A and 1012B. For example, noninflatable positioning member 1016A is connected to injection tube 1014A at locations 1012A and B. Noninflatable positioning members 1016A, 1016B may be connected to injection tubes 1014A, 1014B using any suitable technique, e.g., via a mechanical band, adhesive, welding, or the like.

In some examples, noninflatable positioning members 1016A, 1016B are configured to self-expand from a relatively low-profile configuration (e.g., a relatively linear configuration) to the expanded configuration shown in FIG. 10 when deployed from outer sheath 1004. In addition, or instead, noninflatable positioning members 1016A, 1016B can be configured to be expanded to the expanded configuration shown in FIG. 10 with the aid of a control member, which can include guidewire tube 1002 or a separate control member, such as a push/pull wire.

For example, noninflatable positioning members 1016A, 1016B can be mechanically connected to guidewire tube 1002 at distal portions 1003 of noninflatable positioning member 1016A, 1016B, such that as guidewire tube 1002 moves along longitudinal axis 1015, so does the distal portions 1003 of positioning members 1016A, 1016B. In some of these examples, to expand positioning members 1016A, 1016B radially outwards, a clinician (manually or with the aid of a device) can proximally withdraw guidewire tube 1002 to cause distal portions 1003 of positioning members 1016A, 1016B to move longitudinally towards sheath 1004 and to cause positioning members 1016A, 1016B to expand radially outwards.

Connections at locations 1012A, 1012B to respective injection tubes 1014A, 1014B and connection at distal portion 1003 to guidewire tube 1002 facilitates expansion of noninflatable positioning member 1016A to an expanded configuration when outside of sheath 1004 being pulled proximally while guidewire tube 1002 is pushed distally, e.g., by a clinician.

In some examples, a band (e.g., band 512, FIG. 5) surrounds noninflatable positioning member 1016A, 1016B and injection tubes 1014A, 1014B at each location 1012A and 1012B. Bands at locations 1012 may be of a circumference such that bands can freely traverse longitudinally along respective injection tubes 1014. For example, as the guidewire tube 1002 is pushed distally so that noninflatable positioning members 1016 expand to an expanded configuration, bands may traverse along injection tubes 1014 to facilitate expansion of noninflatable positioning members 1016 to the expanded configuration. Alternatively, the circumference of bands at at least one of locations 1012 may be such that band position along injection tubes 1014 is relatively stationary or that movement longitudinally along injection tubes 1014A, 1014B is minimal. In some examples, noninflatable positioning member 1012A is connected (e.g., via a mechanical band, adhesive, welding, bands, or the like) to injection tube 1014A and is relatively stationary, and noninflatable positioning member 1012B is connected to injection tube 1014B via a band and is free to traverse longitudinally along injection tube 1014B.

In the example shown in FIG. 10, injection tubes 1012A, 1012B are connected to positioning members 1016A, 1016B at midpoints along the length of the injection tubes (measured along the respective longitudinal axes). In other examples, as shown in FIG. 11, injection tubes can be connected to positioning members 1016A, 1016B at their respective distal ends.

FIG. 11 is a conceptual diagram illustrating example catheter system 1100 that includes a guidewire tube 1102, a sheath 1104, injection tubes 1114A and 1114B with respective injection ports 1113A and 1113B, and noninflatable positioning member 1116. Noninflatable positioning member 1116 is similar to one or both of positioning members 1016A, 1016B of FIG. 10, and defines a spiral, helical, or other curvilinear configuration when in an expanded configuration. Catheter system 1100 can include one or more other positioning members in other examples, e.g., as described with respect to FIG. 10.

FIG. 11 illustrates an example expanded configuration of noninflatable positioning member 1116. In examples, noninflatable positioning member 1116 is configured to expand radially outwards towards an expanded configuration and position injection tubes 1114 against vessel wall 104.

Noninflatable positioning member 1116 connects (e.g., via an adhesive, an attachment device, welding, or the like) to two injection tubes 1114A and 1114B at respective locations at or approximately a peak of respective curves 1116A and 1116B of expanded noninflatable positioning member 1116. In some examples, each injection tube 1114 defines a tapered distal end 1112A and 1112B where noninflatable positioning member 1116 connects with injection tubes 1114. Tapered distal ends 1112 may connect with noninflatable positioning member 1116 distally from the peaks of respective curves 1116A and 1116B of noninflatable positioning member 1116.

In some examples, injection ports 1113A and 1113B are positioned at or proximate to curves 1112A and 1112B, respectively. For example, rather than a relatively long longitudinal segment of injection tube contacting vessel wall 104, injection ports 1113A and 1113B and surface of injection tubes 1114A and 1114B proximate to injection ports 1113A and 1113B may contact vessel wall 104.

As with the example of FIG. 10, in some examples, noninflatable positioning members 1116 are configured to self-expand from a relatively low-profile configuration (e.g., a relatively linear configuration) to the expanded configuration shown in FIG. 11 when deployed from outer sheath 1004. In addition, or instead, noninflatable positioning member 1116 can be configured to be expanded to the expanded configuration shown in FIG. 11 with the aid of a control member, which can include guidewire tube 1102 or a separate control member, such as a push/pull wire.

For example, as shown in FIG. 11, guidewire tube 1102 is mechanically connected to noninflatable positioning member 1116 and is configured to cause noninflatable positioning member 1116 to expand radially outward in response to proximal movement. In some examples, noninflatable positioning member 1116 is connected to guidewire tube 1102 at a distal portion 1103 of noninflatable positioning member 1116.

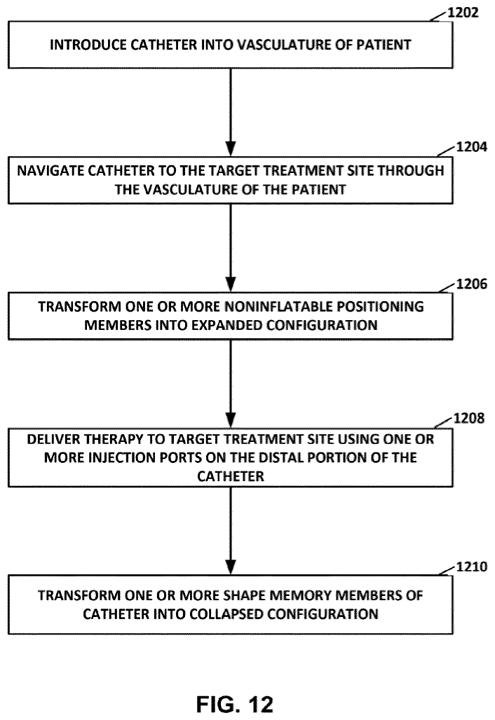

FIG. 12 is a flow diagram illustrating an example process of delivering a therapeutic agent to a patient using a catheter (e.g., catheter system 108) with noninflatable positioning members (e.g., noninflatable positioning members 116) configured to expand to an expanded configuration. While FIG. 12 is primarily described with respect to therapeutic agent delivery to a target treatment site within a renal vessel of a patient, the example processes may be used for other target treatment sites and/or for other cavities within the patient. In addition, while FIG. 12 is primarily described with respect to catheter system 108 of FIG. 1, the method of FIG. 12 can be used with any of the catheters described herein as well as other catheters that include one or more noninflatable positioning members configured to deploy into a radially expanded configuration to position one or more injection ports defined by one or more injection tubes in apposition with a vessel wall.

In some examples, catheter system 108 is disposed in the renal artery and may employ noninflatable positioning members 116 to expand radially outwards towards the target treatment site positioning injection ports 113 of injection tubes 114 in apposition with the target treatment site. This may help increase the likelihood that a therapeutic agent or other fluid will be delivered to intended tissue sites in the patient, successfully penetrate a vessel wall, and counteract any pressure from delivery of therapeutic agent. Catheter system 108 may also reduce an amount of time needed for the clinician to align and orientate catheter system 108 within the vasculature and reduce an overall medical procedure time.

A clinician may introduce catheter system 108 into vasculature of a patient (1202). For example, the clinician may make an incision in the skin of the patient at an insertion site on the patient to reach a blood vessel (e.g., a femoral artery, a radial artery, a brachial artery, or the like) of the patient. The clinician may then insert at least distal portion 108A of catheter system 108 into blood vessel 102 while noninflatable positioning members 116 are in a collapsed configuration. In some examples, noninflatable positioning members 116 and injection tubes 114 are in a collapsed configuration within outer sheath 304 or held in a collapsed configuration by a control member.

The clinician may navigate catheter system 108 to the target treatment site through the vasculature of the patient (1204). The clinician may navigate catheter system 108 using one or more imaging techniques such as X-ray imaging, fluoroscopy, or the like. In some examples, clinician advances catheter system 108 over a guidewire disposed within the vasculature (e.g., via OTW technique, rapid exchange technique, or the like). In some examples, a clinician uses guidewire tube 302 or other control member (e.g., push and/or pull wire) to aid in navigation of catheter system 108 to a target tissue site within the vasculature of the patient. The clinician may advance catheter system 108 within the vasculature until distal portion 108A reaches the target treatment site. In some examples, with regard to renal neuromodulation, the clinician advances catheter system 108 from a trans-radial entry point on the patient to a renal artery of the patient.

The clinician may align injection ports 113 of injection tube(s) 114 with target treatment site, e.g., by advancing or retracting catheter system 108 within the vasculature. A clinician may identify a preferred position of distal portion 108A within vasculature, by identifying a position of a radiopaque marker of catheter system 108.

The clinician may transform noninflatable positioning members 116 of catheter 102 into an expanded configuration (1206). For example, the clinician may proximally withdraw outer sheath 304 that is compressing at least noninflatable positioning members 116 and injection tubes 114 and/or distally push noninflatable positioning members 116 and injection tubes 114 relative to outer sheath 304 to deploy noninflatable positioning members 116 and injection tubes 114 from a distal opening of outer sheath 304. When deployed from outer sheath 304, noninflatable positioning members 116 self-expand radially outwards to help bring injection ports 113 of injection tubes 114 into contact with vessel wall 104. As another example, the clinician may proximally push guidewire tube 302 to cause noninflatable positioning members 116 to expand radially outwards. Alternatively, the clinician may push the guidewire tube 302 distally to cause noninflatable positioning members 116 to expand radially outwards. In some examples, expanding noninflatable positioning members 116 includes, while a first injection tube 114 remains relatively linear and/or stationary, moving a second injection tube 114 in apposition with the wall of blood vessel 104 by at least one noninflatable positioning member 116 of one or more noninflatable positioning members 116.

The clinician may deliver therapy to a target treatment site using one or more injection ports 113 (1208). In some examples, the clinician may actuate fluid delivery system 132 to transmit a therapeutic agent at relatively high pressure (e.g., between 5 MPa to 18 MPa, such as 10 MPa) through injection tube(s) 114 to injection ports 113. In some examples, the injection tube(s) 114 may comprise two injection tubes disposed approximately 180 degrees apart. The pressurized therapeutic agent may exit injection ports 113 and penetrate the wall tissue in apposition with the injection ports 113. In some examples, the therapeutic agent includes a chemical ablation agent. In some examples, with regard to renal neuromodulation, the clinician delivers the therapeutic agent to a portion of vessel wall 104 tissue of the renal artery that faces away from the renal vein. The clinician may continue to deliver the therapeutic agent to the target treatment site until the clinician determines that a user-determined amount of the therapeutic agent has been administered to the patient.

After the delivery of the therapeutic agent, the clinician may transform noninflatable positioning members 116 of catheter system 108 into a collapsed configuration (1210). In some examples, transforming the noninflatable positioning members 116 into the collapse configuration includes folding the noninflatable positioning members 116 radially inwards towards a central longitudinal axis of catheter system 108, e.g., with the aid of outer sheath 304 or guidewire tube 302. In some examples, the clinician pulls proximally on a control member until noninflatable positioning members 116 and injection tubes 114 are substantially collapsed. In addition or instead, the clinician may proximally withdraw catheter 102 into outer sheath 304 or distally push outer sheath 304 over noninflatable positioning members 116 to cause outer sheath 304 to apply compressive force to noninflatable positioning members 116 and injection tubes 114 until noninflatable positioning members 116 and injection tubes 114 are in a collapsed configuration in the lumen of outer sheath 304. Once noninflatable positioning members 116 and injection tubes 114 are in the collapsed configuration, the clinician may remove catheter system 108 from the vasculature and/or navigate distal portion 108A to a second target treatment site within the patient and deliver therapy to the second treatment site via injection ports 113 of injection tubes 114.

The above detailed descriptions of examples of the technology are not intended to be exhaustive or to limit the technology to the precise form disclosed above. Although specific examples of the technology are described above for illustrative purposes, various equivalent modifications are possible within the scope of the technology, as those skilled in the relevant art will recognize. For example, while steps are presented in a given order, alternative examples may perform steps in a different order. The various examples described herein may also be combined to provide further examples. All references cited herein are incorporated by reference as if fully set forth herein.

From the foregoing, it will be appreciated that specific examples of the present disclosure have been described herein for purposes of illustration, but that various modifications may be made without deviating from the present disclosure.

Certain aspects of the present disclosure described in the context of particular examples may be combined or eliminated in other examples. Further, while advantages associated with certain examples have been described in the context of those examples, other examples may also exhibit such advantages, and not all examples need necessarily exhibit such advantages to fall within the scope of the present disclosure. Accordingly, the present disclosure and associated technology can encompass other examples not expressly shown or described herein.

Further, although techniques have been described in which a neuromodulation catheter is positioned at a single location within a single renal artery, in other examples, the neuromodulation catheter may be repositioned to a second treatment site within a single renal artery (e.g., proximal or distal of the first treatment site, may be repositioned in a branch of the single artery, may be repositioned within a different renal vessel on the same side of the patient (e.g., a renal vessel associated with the same kidney of the patient), may be repositioned in a renal vessel on the other side of the patient (e.g., a renal vessel associated with the other kidney of the patient), or any combination thereof. At each location where the neuromodulation catheter is positioned, renal neuromodulation may be performed using any of the techniques described herein or any other suitable renal neuromodulation technique or any combination thereof.

Moreover, unless the word “or” is expressly limited to mean only a single term exclusive from the other items in reference to a list of two or more items, then the use of “or” in such a list is to be interpreted as including (a) any single item in list, (b) all of the items in the list, or (c) any combination of the items in the list. Additionally, the terms “about” or approximately,” when preceding a value, should be interpreted to mean plus or minus 10% of the value, unless otherwise indicated. Additionally, the term “comprising” is used throughout to mean including at least the recited feature(s) such that any greater number of the same feature and/or additional types of other features are not precluded.

Various examples of the disclosure have been described. Any combination of the described systems, operations, or functions is contemplated.

The following examples are a non-limiting list of clauses in accordance with one or more techniques of this disclosure.

Example 1. A catheter comprising: a catheter body defining a central longitudinal axis; one or more noninflatable positioning members connected to the catheter body; and one or more injection tubes disposed on an outer surface of the one or more noninflatable positioning members, each injection tube of the one or more injection tubes defining an injection tube lumen and one or more injection ports in fluid communication with the respective injection tube lumen, wherein the one or more noninflatable positioning members are configured to expand radially outward to position at least one of the one or more injection ports in apposition with a wall of a blood vessel of a patient.

Example 2. The catheter of Example 1, wherein the one or more noninflatable positioning members comprise a shape memory material.

Example 3. The catheter of Example 2, wherein the shape memory material comprises nitinol.

Example 4. The catheter of any of Examples 1-3, wherein each noninflatable positioning member of the one or more noninflatable positioning members is mechanically connected to an injection tube of the one or more injection tubes by a band, the band surrounding the respective noninflatable positioning member and the respective injection tube.

Example 5. The catheter of any of Examples 1-4, wherein the one or more injection tubes comprises two or more injection tubes, and wherein the one or more noninflatable positioning members comprises at least one loop-shaped member radially inwards of the two or more injection tubes.

Example 6. The catheter of Example 5, wherein the one or more noninflatable positioning members comprises an expandable frame, wherein the one or more injection tubes are mechanically connected to the expandable frame, and wherein the at least one loop-shaped member is configured to expand the expandable frame radially outwards.

Example 7. The catheter of Example 5 or Example 6, wherein the at least one loop-shaped member comprises a plurality of loop-shaped members, the plurality of loop-shaped members being longitudinally distributed along the two or more injection tubes.

Example 8. The catheter of any of Examples 5-7, wherein the at least one loop-shaped member is elliptical.

Example 9. The catheter of any of Examples 5-8, wherein the at least one loop-shaped member is configured to collapse to a lower profile state by at least tilting towards the central longitudinal axis.

Example 10. The catheter of any of Examples 1-9, wherein the one or more injection tubes comprise a first injection tube and a second injection tube, and wherein the first injection tube is configured to remain substantially linear and the second injection tube is configured to move away from the first injection tube when the one or more noninflatable positioning members expand radially outward.

Example 11. The catheter of any of Examples 1-4, wherein at least one noninflatable positioning member of the one or more noninflatable positioning members defines a spiral, helical, or sinusoidal structure in the expanded configuration.

Example 12. The catheter of Example 11, wherein the one or more injection tubes and one or more noninflatable positioning members are configured to collapse to a lower profile state by at least tilting towards the central longitudinal axis of the catheter body when a control member is pushed distally.

Example 13. The catheter of Example 12, wherein the one or more noninflatable positioning members are configured to expand radially outward when the control member is pulled proximally.

Example 14. The catheter of any of Examples 1-13, wherein the one or more noninflatable positioning members are further configured to hold the one or more injection ports against the vessel wall during high-pressure injection of a chemical through the injection lumens and out the one or more injection ports.

Example 15. The catheter of Example 1, wherein the one or more noninflatable positioning members are each within the one or more injection tubes.

Example 16. The catheter of any of Examples 1-15, wherein at least a portion of at least one injection tube of the one or more injection tubes is flexible.

Example 17. The catheter of any of Examples 1-16, wherein the one or more injection tubes comprise multiple injection tubes and the injection ports of the multiple injection tubes are longitudinally aligned.

Example 18. The catheter of any of Examples 1-17, wherein the one or more noninflatable positioning members are configured to self-expand.

Example 19. The catheter of any of Examples 1-18, wherein the one or more injection tubes comprises two injection tubes disposed 180 degrees apart.

Example 20. The catheter of any of Examples 1-19, wherein the catheter body defines a catheter lumen, the catheter further comprising a fluid delivery tube disposed within the catheter lumen, wherein each injection tube of the one or more injection tubes comprises a proximal portion disposed within the catheter lumen and in fluid communication with the fluid delivery tube.

Example 21. The catheter of any of Examples 1-20, wherein the one or more injection tubes comprises a plurality of injection tubes, and wherein each injection tube of the plurality of injection tubes exits the catheter lumen and extends along an outer surface of the catheter body proximal to a proximal end of the one or more noninflatable positioning members.

Example 22. The catheter of any of Examples 1-21, wherein the catheter body defines a catheter lumen, the catheter further comprising a control member disposed within the catheter lumen, the control member connected to the one or more noninflatable positioning members and being configured to cause the one or more noninflatable positioning members to expand radially outward.

Example 23. The catheter of Example 22, wherein the control member comprises a guidewire tube defining a guidewire lumen configured to receive a guidewire.

Example 24. The catheter of any of Example 22 or Example 23, wherein the control member comprises one or more pull wires disposed within the catheter lumen, wherein the one or more noninflatable positioning members are configured to expand radially outward in response to proximal movement of at least one pull wire of the one or more pull wires

Example 25. A method comprising: navigating a catheter through the vasculature of a patient to a target treatment site, wherein the catheter comprises: a catheter body; one or more noninflatable positioning members connected to the catheter body; and one or more injection tubes disposed on an outer surface of the one or more noninflatable positioning members, each of the injection tubes of the one or more injection tubes defining an injection tube lumen and one or more injection ports in fluid communication with the respective injection tube lumen; deploying the one or more noninflatable positioning members to an expanded configuration to position at least one of the one or more injection ports in apposition with a wall of a blood vessel of a patient; and delivering therapy to the target treatment site via the one or more injection ports of the one or more injection tubes Example 26. The method of Example 25, wherein delivering the therapy to the target treatment site comprises delivering a therapeutic agent to the tissue of the patient via the one or more injection ports of the one or more injection tubes.

Example 27. The method of Example 26, wherein the therapeutic agent comprises a chemical ablation agent.

Example 28. The method of any of Examples 25-27, wherein the one or more injection tubes comprises two injection tubes disposed 180 degrees apart.

Example 29. The method of any of Examples 25-28, further comprising transforming the one or more noninflatable positioning members from the expanded configuration to the collapsed configuration, wherein transforming the one or more noninflatable positioning members from the expanded configuration to the collapsed configuration comprises collapsing the one or more noninflatable positioning members radially inwards towards a central longitudinal axis of the catheter body.

Example 30. The method of any of Examples 25-29, wherein the catheter body defines a catheter lumen, the catheter further comprising a control member disposed within the catheter lumen, the control member connected to the one or more noninflatable positioning members, wherein expanding the one or more noninflatable positioning members comprises: proximally pulling the control member to expand the one or more noninflatable positioning members radially outward.

Example 31. The method of Example 30, wherein the control member comprises a guidewire tube defining a guidewire lumen configured to receive a guidewire.

Example 32. The method of any of Examples 25-31, wherein the target treatment site comprises a first target treatment site, the method further comprising:
transforming the one or more noninflatable positioning members from the expanded configuration to a collapsed configuration; navigating the catheter through the vasculature of the patient to a second target treatment site; deploying the one or more noninflatable positioning members from the collapsed configuration to the expanded configuration at the second target treatment site; and delivering the therapy to the second target treatment site via the one or more injection ports of the one or more injection tubes.

Example 33. The method of any of Examples 25-32, wherein the one or more noninflatable positioning members comprise a shape memory material.

Example 34. The method of any of Examples 25-33, wherein the one or more injection tubes comprise a first injection tube and a second injection tube, wherein expanding the one or more noninflatable positioning members comprises, while the first injection tube remains substantially stationary, deploying at least one noninflatable positioning member of the one or more noninflatable positioning members to move the second injection tube away from the first injection tube.

Example 35. A catheter comprising: a catheter body defining a catheter lumen and a central longitudinal axis; an inner member disposed within the catheter lumen; one or more noninflatable positioning members; and one or more injection tubes connected to the one or more noninflatable positioning members, each injection tube of the one or more injection tubes defining an injection tube lumen and one or more injection ports in fluid communication with the respective injection tube lumen, wherein in response to movement of the inner member relative to the catheter body, the one or more noninflatable positioning members and the one or more injection tubes are configured to move radially outwards away from the central longitudinal axis.

Example 36. The catheter of Example 35, wherein the one or more noninflatable positioning members comprise a shape memory material.

Example 37. The catheter of Example 35 or Example 36, wherein the one or more injection tubes comprise a first injection tube and a second injection tube, and wherein the first injection tube is configured to remain substantially linear while the second injection tube moves radially away from the first injection tube when the one or more noninflatable positioning members move radially outwards away from the central longitudinal axis.

Example 38. The catheter of Examples 35-37, wherein at least one noninflatable positioning member of the one or more noninflatable positioning members defines a spiral, helical, or sinusoidal structure in the expanded state.

Further disclosed herein is the subject-matter of the following clauses:
1. A catheter comprising:
   a catheter body defining a central longitudinal axis;
   one or more noninflatable positioning members connected to the catheter body; and
   one or more injection tubes disposed on an outer surface of the one or more noninflatable positioning members, each injection tube of the one or more injection tubes defining an injection tube lumen and one or more injection ports in fluid communication with the respective injection tube lumen,
   wherein the one or more noninflatable positioning members are configured to expand radially outward to position at least one of the one or more injection ports in apposition with a wall of a blood vessel of a patient.
2. The catheter of clause 1, wherein the one or more noninflatable positioning members comprise a shape memory material.
3. The catheter of clause 2, wherein the shape memory material comprises nitinol.
4. The catheter of any of clauses 1-3, wherein each noninflatable positioning member of the one or more noninflatable positioning members is mechanically connected to an injection tube of the one or more injection tubes by a band, the band surrounding the respective noninflatable positioning member and the respective injection tube.
5. The catheter of any of clauses 1-4, wherein the one or more injection tubes comprises two or more injection tubes, and wherein the one or more noninflatable positioning members comprises at least one loop-shaped member radially inwards of the two or more injection tubes.
6. The catheter of clause 5, wherein the one or more noninflatable positioning members comprises an expandable frame, wherein the one or more injection tubes are mechanically connected to the expandable frame, and wherein the at least one loop-shaped member is configured to expand the expandable frame radially outwards.
7. The catheter of clause 5 or clause 6, wherein the at least one loop-shaped member comprises a plurality of loop-shaped members, the plurality of loop-shaped members being longitudinally distributed along the two or more injection tubes.
8. The catheter of any of clauses 5-7, wherein the at least one loop-shaped member is elliptical.
9. The catheter of any of clauses 5-8, wherein the at least one loop-shaped member is configured to collapse to a lower profile state by at least tilting towards the central longitudinal axis.
10. The catheter of any of clauses 1-9, wherein the one or more injection tubes comprise a first injection tube and a second injection tube, and wherein the first injection tube is configured to remain substantially linear and the second injection tube is configured to move away from the first injection tube when the one or more noninflatable positioning members expand radially outward.
11. The catheter of any of clauses 1-4, wherein at least one noninflatable positioning member of the one or more noninflatable positioning members defines a spiral, helical, or sinusoidal structure in the expanded configuration.
12. The catheter of clause 11, wherein the one or more injection tubes and one or more noninflatable positioning members are configured to collapse to a lower profile state by at least tilting towards the central longitudinal axis of the catheter body when a control member is pushed distally.
13. The catheter of clause 12, wherein the one or more noninflatable positioning members are configured to expand radially outward when the control member is pulled proximally.
14. The catheter of any of clauses 1-13, wherein the one or more noninflatable positioning members are further configured to hold the one or more injection ports against the vessel wall during high-pressure injection of a chemical through the injection lumens and out the one or more injection ports.
15. The catheter of any of clauses 1-14, wherein the one or more noninflatable positioning members are configured to self-expand.

1. A catheter comprising:
a catheter body defining a central longitudinal axis;
one or more noninflatable positioning members connected to the catheter body; and
one or more injection tubes disposed on an outer surface of the one or more noninflatable positioning members, each injection tube of the one or more injection tubes defining an injection tube lumen and one or more injection ports in fluid communication with the respective injection tube lumen,
wherein the one or more noninflatable positioning members are configured to expand radially outward to position at least one of the one or more injection ports in apposition with a wall of a blood vessel of a patient.

2. The catheter of claim 1, wherein the one or more noninflatable positioning members comprise a shape memory material.

3. (canceled)

4. The catheter of claim 1, wherein each noninflatable positioning member of the one or more noninflatable positioning members is mechanically connected to an injection tube of the one or more injection tubes by a band, the band surrounding the respective noninflatable positioning member and the respective injection tube.

5. The catheter of claim 1, wherein the one or more injection tubes comprises two or more injection tubes, and wherein the one or more noninflatable positioning members comprises at least one loop-shaped member radially inwards of the two or more injection tubes.

6. The catheter of claim 5, wherein the one or more noninflatable positioning members comprises an expandable frame, wherein the one or more injection tubes are mechanically connected to the expandable frame, and wherein the at least one loop-shaped member is configured to expand the expandable frame radially outwards.

7. The catheter of claim 5, wherein the at least one loop-shaped member comprises a plurality of loop-shaped members, the plurality of loop-shaped members being longitudinally distributed along the two or more injection tubes.

8. (canceled)

9. The catheter of claim 5, wherein the at least one loop-shaped member is configured to collapse to a lower profile state by at least tilting towards the central longitudinal axis.

10. The catheter of claim 1, wherein the one or more injection tubes comprise a first injection tube and a second injection tube, and wherein the first injection tube is configured to remain substantially linear and the second injection tube is configured to move away from the first injection tube when the one or more noninflatable positioning members expand radially outward.

11. The catheter of claim 1, wherein at least one noninflatable positioning member of the one or more noninflatable positioning members defines a spiral, helical, or sinusoidal structure in the expanded configuration.

12. The catheter of claim 11, wherein the one or more injection tubes and one or more noninflatable positioning members are configured to collapse to a lower profile state by at least tilting towards the central longitudinal axis of the catheter body when a control member is pushed distally.

13. The catheter of claim 12, wherein the one or more noninflatable positioning members are configured to expand radially outward when the control member is pulled proximally.

14. The catheter of claim 1, wherein the one or more noninflatable positioning members are further configured to hold the one or more injection ports against the vessel wall during high-pressure injection of a chemical through the injection lumens and out the one or more injection ports.

15. (canceled)

16. A method comprising:
navigating a catheter through the vasculature of a patient to a target treatment site, wherein the catheter comprises:
a catheter body;
one or more noninflatable positioning members connected to the catheter body; and
one or more injection tubes disposed on an outer surface of the one or more noninflatable positioning members, each of the injection tubes of the one or more injection tubes defining an injection tube lumen and one or more injection ports in fluid communication with the respective injection tube lumen;
deploying the one or more noninflatable positioning members to an expanded configuration to position at least one of the one or more injection ports in apposition with a wall of a blood vessel of a patient; and
delivering therapy to the target treatment site via the one or more injection ports of the one or more injection tubes.

17. The method of claim 16, wherein delivering the therapy to the target treatment site comprises delivering a therapeutic agent to the tissue of the patient via the one or more injection ports of the one or more injection tubes.

18. The method of claim 16, further comprising transforming the one or more noninflatable positioning members from the expanded configuration to the collapsed configuration,
wherein transforming the one or more noninflatable positioning members from the expanded configuration to the collapsed configuration comprises collapsing the one or more noninflatable positioning members radially inwards towards a central longitudinal axis of the catheter body.

19. The method of claim 16, wherein the catheter body defines a catheter lumen, the catheter further comprising a control member disposed within the catheter lumen, the control member connected to the one or more noninflatable positioning members, wherein expanding the one or more noninflatable positioning members comprises:
proximally pulling the control member to expand the one or more noninflatable positioning members radially outward.

20. The method of claim 16, wherein the target treatment site comprises a first target treatment site, the method further comprising:
transforming the one or more noninflatable positioning members from the expanded configuration to a collapsed configuration;
navigating the catheter through the vasculature of the patient to a second target treatment site;
deploying the one or more noninflatable positioning members from the collapsed configuration to the expanded configuration at the second target treatment site; and
delivering the therapy to the second target treatment site via the one or more injection ports of the one or more injection tubes.

21. A catheter comprising:
a catheter body defining a catheter lumen and a central longitudinal axis;
an inner member disposed within the catheter lumen;
one or more noninflatable positioning members; and
one or more injection tubes connected to the one or more noninflatable positioning members, each injection tube of the one or more injection tubes defining an injection tube lumen and one or more injection ports in fluid communication with the respective injection tube lumen,
wherein in response to movement of the inner member relative to the catheter body, the one or more noninflatable positioning members and the one or more injection tubes are configured to move radially outwards away from the central longitudinal axis.

22. The catheter of claim 21, wherein the one or more injection tubes comprise a first injection tube and a second injection tube, and wherein the first injection tube is configured to remain substantially linear while the second injection tube moves radially away from the first injection tube when the one or more noninflatable positioning members move radially outwards away from the central longitudinal axis.

23. The catheter of claim 21, wherein at least one noninflatable positioning member of the one or more noninflatable positioning members defines a spiral, helical, or sinusoidal structure in the expanded state.
